(19) United States
(12) Patent Application Publication
(10) Pub. No.: US 2015/0234206 A1
(43) Pub. Date: Aug. 20, 2015

(54) CONFIGURABLE ADAPTIVE OPTICAL MATERIAL AND DEVICE

(71) Applicants: Vincent James Lee, San Francisco, CA (US); Aparana Reddy Aryabumi, San Jose, CA (US); Sheila Nabanja, San Jose, CA (US)

(72) Inventors: Vincent James Lee, San Francisco, CA (US); Aparana Reddy Aryabumi, San Jose, CA (US); Sheila Nabanja, San Jose, CA (US)

(73) Assignee: AliphCom, San Francisco, CA (US)

(21) Appl. No.: 14/313,901

(22) Filed: Jun. 24, 2014

Related U.S. Application Data
(63) Continuation-in-part of application No. 14/183,463, filed on Feb. 18, 2014.

(51) Int. Cl. G02C 7/08; A61B 3/00; A61B 3/14; G02C 7/02; G02C 11/00

(52) U.S. Cl. CPC G02C 7/085; G02C 7/022; G02C 11/10; A61B 3/14; A61B 3/0025; G02C 2202/16

(57) ABSTRACT

Techniques associated with an adaptive optical material and device are described, including a device having a layer, a substrate, and an intermediate layer disposed between the substrate and the layer. The intermediate layer may include a first bladder and a second bladder. Each bladder may have a surface portion associated with an intermediate layer and another surface portion associated with a substrate. A first bladder may receive a first volume of fluid to form a first distance between the surface portions, and a first bladder may receive a second volume of fluid to form a second distance between the surface portions. A portion of a surface of the layer may include a degree of curvature relative to a line perpendicular to the substrate based on a difference between the first distance and the second distance. The degree of curvature may focus a subset of collimated light rays substantially at a point.

### CROSS REFERENCE TO RELATED APPLICATIONS

This application is a continuation in part of U.S. application Ser. No. 14/183,463, filed Feb. 18, 2014, which is hereby incorporated by reference in its entirety for all purposes.

### FIELD

The present application relates generally to portable electronics, wearable electronics, consumer electronics, electronic systems, optical systems and more specifically to systems, electronics, structures and methods for optical correction, display and control systems. More specifically, a configurable optical lens is formed to modify its optical characteristics in real time (or near real time).

### BACKGROUND

As more electronic devices include displays that present information, images, icons, text, GUI's, notifications, numerals, and the like, many users find themselves having to diver their attention to a display tied to a particular device (e.g., a tablet, pad, smartphone, laptop, wireless client device, media device, etc.) in order to divine information being presented by that device and/or to interact with the device to implement commands or other actions using a GUI, cursor, gesture recognition, or the like. In other scenarios a user may wear a portable display (e.g., virtual reality display/glasses/headset or smart glasses, etc.) that present information to the user via the eyes, typically in a virtual image or images projected into the eye.

In some examples, those images are presented to a single eye, and in other examples the images are presented to both eyes; however, some users may use corrective eyewear or contacts lenses to correct for nearsightedness, farsightedness, and/or other aberrations associated with the eye. Therefore, a user may be compelled to get prescription lenses to correct vision for normal activities such as reading, working, driving, etc.

Conventionally, optical correction is typically provided by using eyewear having lenses made to have a given power (e.g., in diopters) for aberrations of a user's eyes. However, aberrations of an eye may change over time, or the eyewear may be shared amongst several users who have different aberrations.

Thus, what is needed is a solution for providing optical correction, diagnoses, and correction of aberrations or disease of the eye without the limitations of conventional techniques.

### BRIEF DESCRIPTION OF THE DRAWINGS

Various embodiments or examples (“examples”) of the present application are disclosed in the following detailed description and the accompanying drawings. The drawings are not necessarily to scale:

FIG. 1A depicts a front view of one example of a wearable device including adaptive optics, according to an embodiment;

FIG. 1B depicts a side view of one example of a first display system and delivery optics for a wearable device including adaptive optics, according to an embodiment;

FIG. 1C depicts a side view of one example of a second display system and delivery optics for a wearable device including adaptive optics, according to an embodiment;

FIG. 1D depicts cross-sectional views of first and second delivery optics in optical communication with first and second adaptive optics, respectively, according to an embodiment;

FIG. 2 depicts an exemplary computer system, according to an embodiment;

FIG. 3A depicts a cross-sectional view of one example of adaptive optics coupled with a control system, according to an embodiment;

FIG. 3B depicts a cross-sectional view of one example of a control system modifying index of refraction of adaptive optics, according to an embodiment;

FIG. 3C depicts a cross-sectional view of another example of adaptive optics coupled with a control system, according to an embodiment;

FIG. 3D depicts a cross-sectional view of another example of a control system modifying index of refraction of adaptive optics, according to an embodiment;

FIG. 4A depicts a cross-sectional view of one example of an eye in optical communication with delivery optics and adaptive optics, according to an embodiment;

FIG. 4B depicts a cross-sectional view of one example of direct and projected images presented to an eye in optical communication with delivery optics and adaptive optics, according to an embodiment;

FIG. 5 depicts a cross-sectional view of yet another example of adaptive optics coupled with a control system operative to modify an index of refraction of the adaptive optics, according to an embodiment;

FIG. 6 depicts a cross-sectional view of still another example of adaptive optics coupled with a control system operative to modify an index of refraction of the adaptive optics, according to an embodiment;

FIG. 7 depicts a block diagram of one example of a display system optically coupled with an eye through delivery optics, according to an embodiment;

FIG. 8A depicts one example of a model image projected into an eye by a delivery system, according to an embodiment;

FIG. 8B depicts another example of a of a model image projected into an eye by a delivery system, according to an embodiment;

FIG. 9 depicts one example of sensing image displacement for vision correction using adaptive optics, according to an embodiment;

FIGS. 10A and 10B depict a cross-sectional view of an example of an adaptive optical material using one or more fluids, according to an embodiment;

FIGS. 11A and 11B depict top views of examples of an adaptive optical material using one or more fluids, according to an embodiment;

FIG. 12 depicts an example of an adaptive optical material using one or more fluids coupled to a control, according to an embodiment;

FIGS. 13A and 13B depict cross-sectional views of examples of an adaptive optical material having a shape modified by one or more fluids, according to an embodiment;

FIG. 14 depicts a cross-sectional view of an example of an adaptive optical material using capacitance, according to an embodiment;

FIG. 15 depicts a cross-sectional view of an example of an adaptive optical material using electroactive material, according to an embodiment;

FIG. 16 depicts a top view of an example of an adaptive optical material using an electrode grid, according to an embodiment; and

FIG. 17 depicts a computer system suitable for use with an adaptive optical material, according to an embodiment.

### DETAILED DESCRIPTION

Various embodiments or examples may be implemented in numerous ways, including as a system, a process, an apparatus, a user interface, or a series of program instructions on a non-transitory computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying drawing FIGS. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

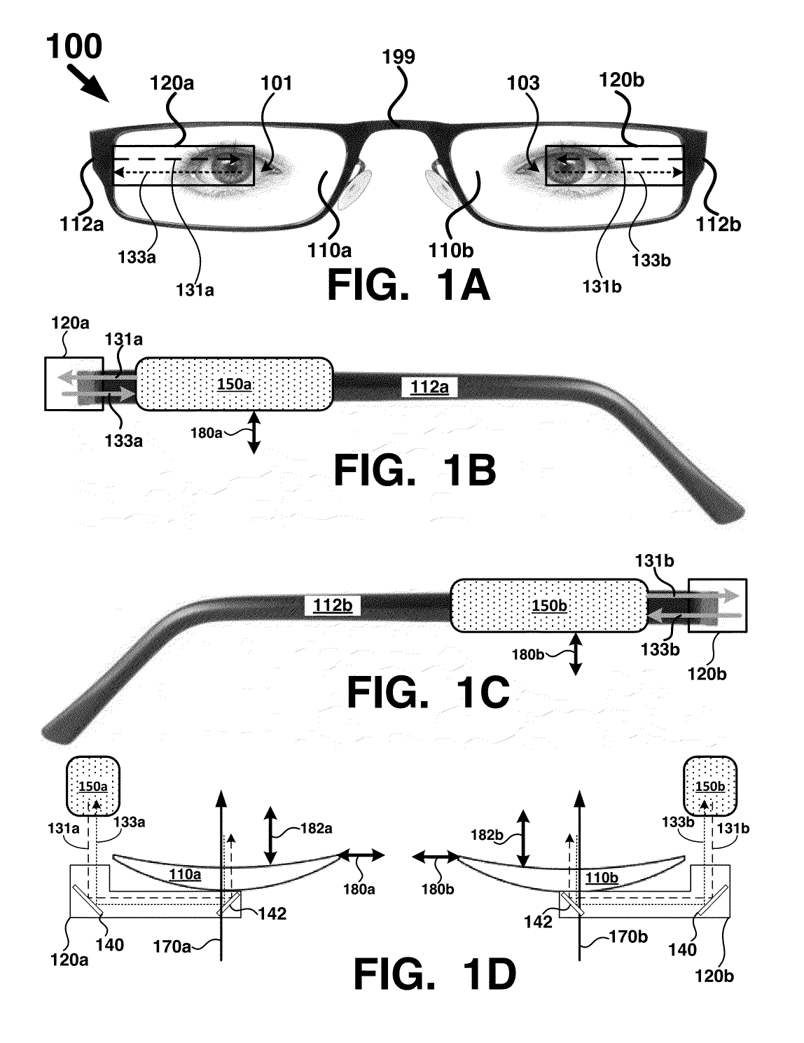

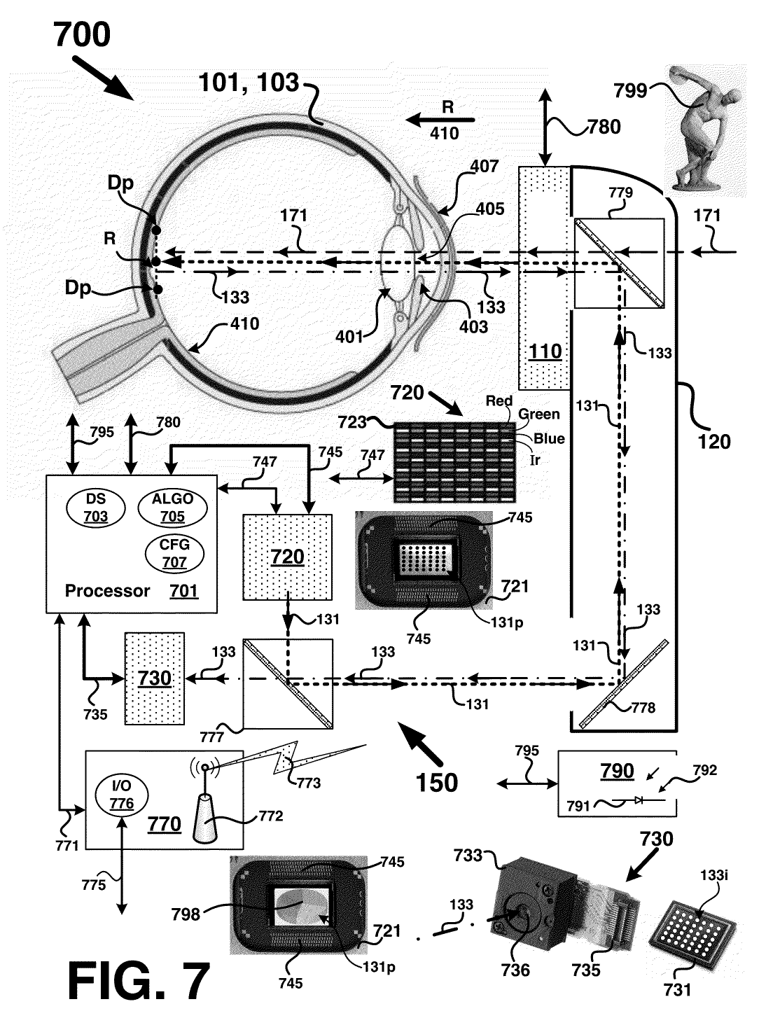

FIG. 1A depicts a front view of one example of a wearable device 100 including adaptive optics 110a and 110b. Adaptive optics 110a and/or 110b may be mounted to a frame, housing, helmet, structure, or other and will be denoted herein as chassis 199. The actual chassis 199 may be application dependent and is not limited to the exampled depicted herein. For purposes of explanation, chassis 199 depicted in FIG. 1A may be a frame for eyeglasses and may include adaptive optics 110a and 110b mounted to rims of chassis 199. Temples 112a and 112b may be movably connected with chassis 199 using hinges (not shown), fixedly connected with chassis 199 or flexibly mounted with chassis 199, for example. Wearable device 100 may further include delivery optics 120a and/or 120b that are positioned relative to adaptive optics (110a, 110b) so that an image 131a and/or 131b that is optically coupled by the delivery optics (120a, 120b) for visual sensing by eyes 101 and/or 103 of a user (not shown) may be focused on a retina of the eyes (101, 103) by first being optically coupled with the adaptive optics (110a, 110b) so that the image (131a, 131b) passes through the adaptive optics (110a, 110b), is focused by the adaptive optics (110a, 110b) onto the retina of each eye (101, 103) along with external visual images (e.g., ambient images in a field of view and/or within visual perception of the user's eyes 101 and/or 103), such as scenery or other images as will be described in greater detail below. Delivery optics (120a, 120b) may also optically coupled reflected light 133a and/or 133b that is reflected off of the retina, back through the lens and pupil of the eyes (101, 103) and into the delivery optics (120a, 120b) where the reflected light may be optically coupled with an image sensor system as will be described in greater detail below. In some examples, wearable device 100 may only service one eye of a user and may only include a single adaptive optics, and a single delivery optic. Delivery optics 120a and/or 120b may be coupled with temples 112a and/or 112b respectively. Reflected light 133 (e.g., 133a, 133b) may be regarded as a retinal reflection image of a projected image 131 (e.g., 131a, 131b) that is incident on a retina and at least a portion of the projected image is reflected off of the retina and optically coupled through the adaptive optics 110 (e.g., 110a, 110b) and into delivery optics 120. An image projector for projecting the image that comprises projected image 131 and an image capture device for capturing the reflected light that comprises the retinal reflection image 133 will be described in greater detail below in regards to FIGS. 7-9.

FIG. 1B depicts a side view of one example of a first display system 150a and delivery optics 120a for a wearable device 100 including adaptive optics 110a. Whereas, FIG. 1C depicts a side view of one example of a second display system 150b and delivery optics 120b for a wearable device 100 including adaptive optics 110b. Here, first and second display systems 150a and 150b may service right and left eyes 101 and 103 respectively and their associated adaptive optics 110a and 110b, for example. Display systems (150a, 150b) may include a display and optics (e.g., a PICO projector) for projecting or otherwise optically coupling images (131a, 131b) from the display to right and/or left eyes 101 and 103 using delivery optics (120a, 120b). Display systems (150a, 150b) may include an image capture device (e.g., a digital camera, digital video capture system, CCD image sensor, CMOS image sensor, etc.) for capturing images in reflected light (133a, 133b) that is optically coupled with the image capture device via delivery optics (120a, 120b). Display systems (150a, 150b) may be coupled with temples 112a and/or 112b respectively and may be positioned relative to their respective delivery optics (120a, 120b) to optically couple projected images 131a and 131b with the delivery optics (120a, 120b) and to receive reflected images 133a and 133b that are optically coupled with the display systems (150a, 150b) by the delivery optics (120a, 120b).

Display systems (150a, 150b) may transmit and/or receive one or more signals 180a and/or 180b that may be operative to control adaptive optics 110a and/or 110b. As will be described in greater detail below, signals 180a and/or 180b may be operative to change an index of refraction and/or a focal length of adaptive optics 110a and/or 110b so that images (131a, 131b) and ambient images 170a and 170b (see FIG. 1D) (e.g., those not generated by 150a, 150b) may be focused on the retinas of eyes 101 and/or 103. Changes in index of refraction and/or a focal length of adaptive optics 110a and/or 110b may occur in real time.

Moving down now to FIG. 1D are cross-sectional views of first and second delivery optics (120a, 120b) in optical communication (131a, 133a, 131b, 133b) with first and second adaptive optics (110a, 110b), respectively. First and second delivery optics (120a, 120b) may include optical components and systems operative to optically couple images between one or more of the eyes (101, 103), adaptive optics (110a, 110b), or display systems (150a, 150b). For example, first and second delivery optics (120a, 120b) may include mirror 140 and 142 to direct light comprising images (131a, 133a, 131b, 133b) between one or more of the eyes (101, 103), adaptive optics (110a, 110b), or display systems (150a, 150b). Other optical components including but not limited to lenses, aspheric lenses, prisms, fiber optics, lens arrays, polarizing optics, and Fresnel lenses, just to name a few. The examples of adaptive optics (110a, 110b), display systems (150a, 150b) or deliver optics depicted herein are non-limiting examples presented for purposes of illustrating the present application.

Signals 180a and/or 180b from display systems (150a, 150b) or from some other system or processor are in electrical communication (e.g., wired or wireless communications) with adaptive optics (110a, 110b) and may be operative to change (182a, 182b) one or more parameters of adaptive optics (110a, 110b) including but not limited to focal length, index of refraction, and shape, just to name a few. Here, ambient light (170a, 170b) passes through delivery optics (120a, 120b), adaptive optics (110a, 110b) or both and into the eyes (101, 103) where it may impinge on the retina as a focused image, an out of focus image (e.g., myopia—nearsightedness or hyperopia—farsightedness) or blurry image (e.g., astigmatism due to distortion of the cornea). Projected images 131a and/or 131b and/or reflected images 133a and/or 133b may be incident on retinas of the eyes 101 and/or 103 along with the ambient images 170a and/or 170b. Adaptive optics (110a, 110b) may be operative to bring both ambient (170a, 170b) and projected (131a, 131b) images into focus on the retinas of the eyes (101, 103). In some examples, display systems (150a, 150b) may be collective referred to as display system 150 or control system 150.

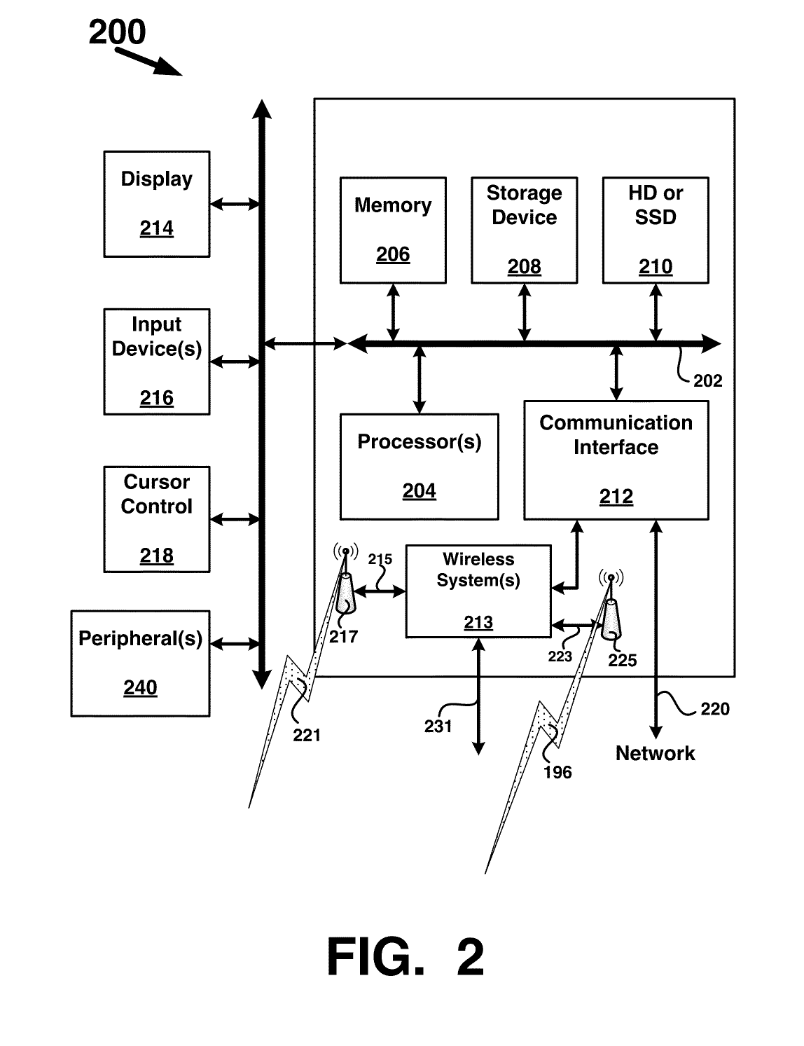

FIG. 2 depicts an exemplary computer system 200 suitable for use in the systems, methods, and apparatus described herein that include hybrid display 102. In some examples, computer system 200 may be used to implement circuitry, computer programs, applications (e.g., APP's), configurations (e.g., CFG's), methods, processes, or other hardware and/or software to implement techniques described herein. Computer system 200 includes a bus 202 or other communication mechanism for communicating information, which interconnects subsystems and devices, such as one or more processors 204, system memory 206 (e.g., RAM, SRAM, DRAM, Flash), storage device 208 (e.g., Flash Memory, ROM), disk drive 210 (e.g., magnetic, optical, solid state), communication interface 212 (e.g., modem, Ethernet, one or more varieties of IEEE 802.11, WiFi, WiMAX, WiFi Direct, Bluetooth, Bluetooth Low Energy, NFC, Ad Hoc WiFi, HackRF, USB-powered software-defined radio (SDR), WAN or other), display 214 (e.g., CRT, LCD, OLED, touch screen), one or more input devices 216 (e.g., keyboard, stylus, touch screen display), cursor control 218 (e.g., mouse, trackball, stylus), one or more peripherals 240. Some of the elements depicted in computer system 200 may be optional, such as elements 214-218 and 240, for example and computer system 200 need not include all of the elements depicted.

According to some examples, computer system 200 performs specific operations by processor 204 executing one or more sequences of one or more instructions stored in system memory 206. Such instructions may be read into system memory 206 from another non-transitory computer readable medium, such as storage device 208 or disk drive 210 (e.g., a HD or SSD). In some examples, circuitry may be used in place of or in combination with software instructions for implementation. The term “non-transitory computer readable medium” refers to any tangible medium that participates in providing instructions to processor 204 for execution. Such a medium may take many forms, including but not limited to, non-volatile media and volatile media. Non-volatile media includes, for example, Flash Memory, optical, magnetic, or solid state disks, such as disk drive 210. Volatile media includes dynamic memory (e.g., DRAM), such as system memory 206. Common forms of non-transitory computer readable media includes, for example, floppy disk, flexible disk, hard disk, Flash Memory, SSD, magnetic tape, any other magnetic medium, CD-ROM, DVD-ROM, Blu-Ray ROM, USB thumb drive, SD Card, any other optical medium, punch cards, paper tape, any other physical medium with patterns of holes, RAM, PROM, EPROM, FLASH-EPROM, any other memory chip or cartridge, or any other medium from which a computer may read.

Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 202 for transmitting a computer data signal. In some examples, execution of the sequences of instructions may be performed by a single computer system 200. According to some examples, two or more computer systems 200 coupled by communication link 220 (e.g., LAN, Ethernet, PSTN, wireless network, WiFi, WiMAX, Bluetooth (BT), NFC, Ad Hoc WiFi, HackRF, USB-powered software-defined radio (SDR), or other) may perform the sequence of instructions in coordination with one another. Computer system 200 may transmit and receive messages, data, and instructions, including programs, (e.g., application code), through communication link 220 and communication interface 212. Received program code may be executed by processor 204 as it is received, and/or stored in a drive unit 210 (e.g., a SSD or HD) or other non-volatile storage for later execution. Computer system 200 may optionally include one or more wireless systems 213 in communication with the communication interface 212 and coupled (215, 223) with one or more antennas (217, 225) for receiving and/or transmitting RF signals (221, 196), such as from a WiFi network, BT radio, or other wireless network and/or wireless devices, devices 100, 100c, 100d, 100e, for example. Examples of wireless devices include but are not limited to: a data capable strap band, wristband, wristwatch, digital watch, or wireless activity monitoring and reporting device; a smartphone; cellular phone; tablet; tablet computer; pad device (e.g., an iPad); touch screen device; touch screen computer; laptop computer; personal computer; server; personal digital assistant (PDA); portable gaming device; a mobile electronic device; and a wireless media device, just to name a few. Computer system 200 in part or whole may be used to implement one or more systems, devices, or methods that communicate with device 100 via RF signals (e.g., 196) or a hard wired connection (e.g., data port). For example, a radio (e.g., a RF receiver) in wireless system(s) 213 may receive transmitted RF signals (e.g., 196 or other RF signals) from device 100 that include one or more datum (e.g., sensor system information, content, data, or other). Computer system 200 in part or whole may be used to implement a remote server or other compute engine in communication with systems, devices, or method for use with the device 100 or other devices as described herein. Computer system 200 in part or whole may be included in a portable device such as a wearable display (e.g., wearable display 100) smartphone, media device, wireless client device, tablet, or pad, for example.

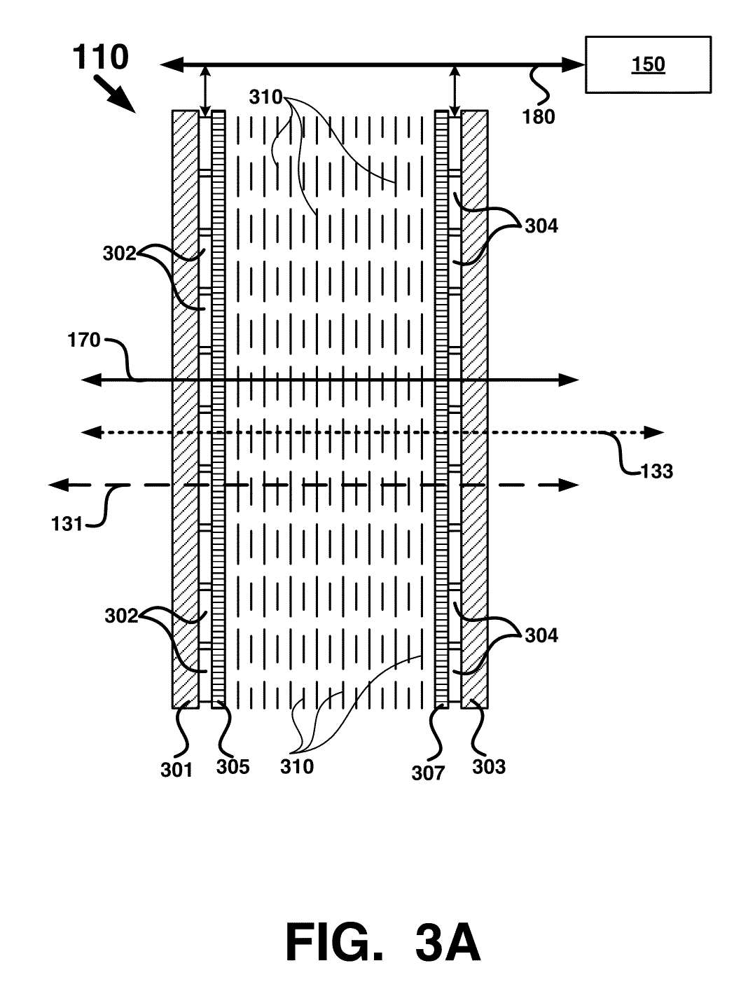

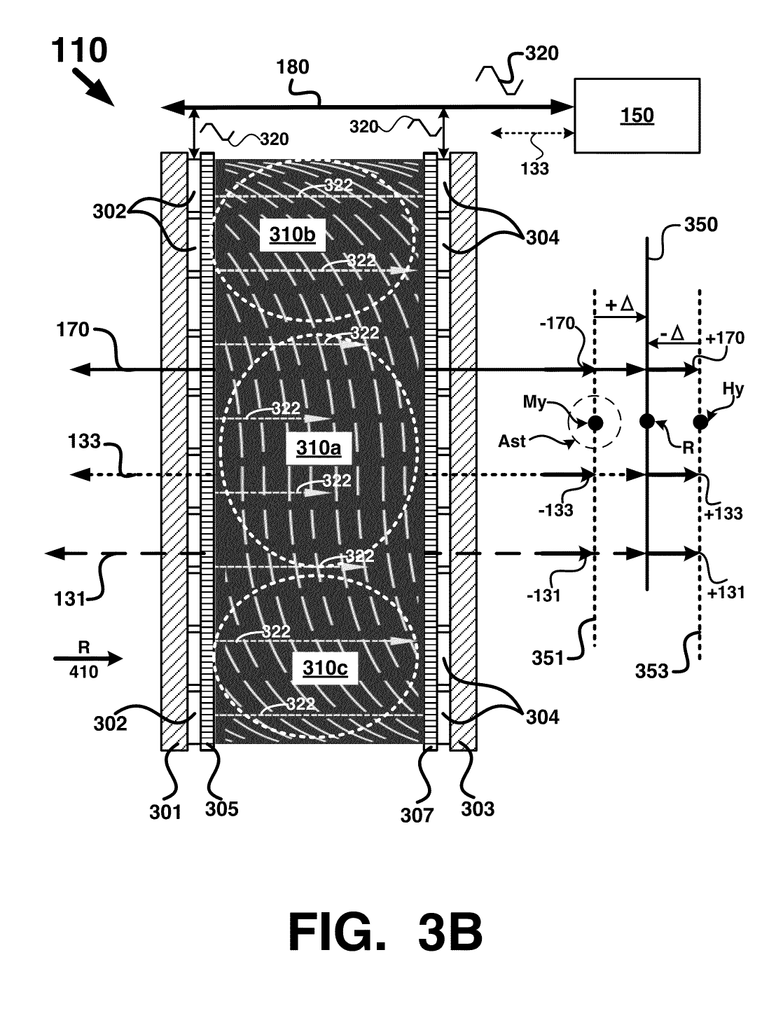

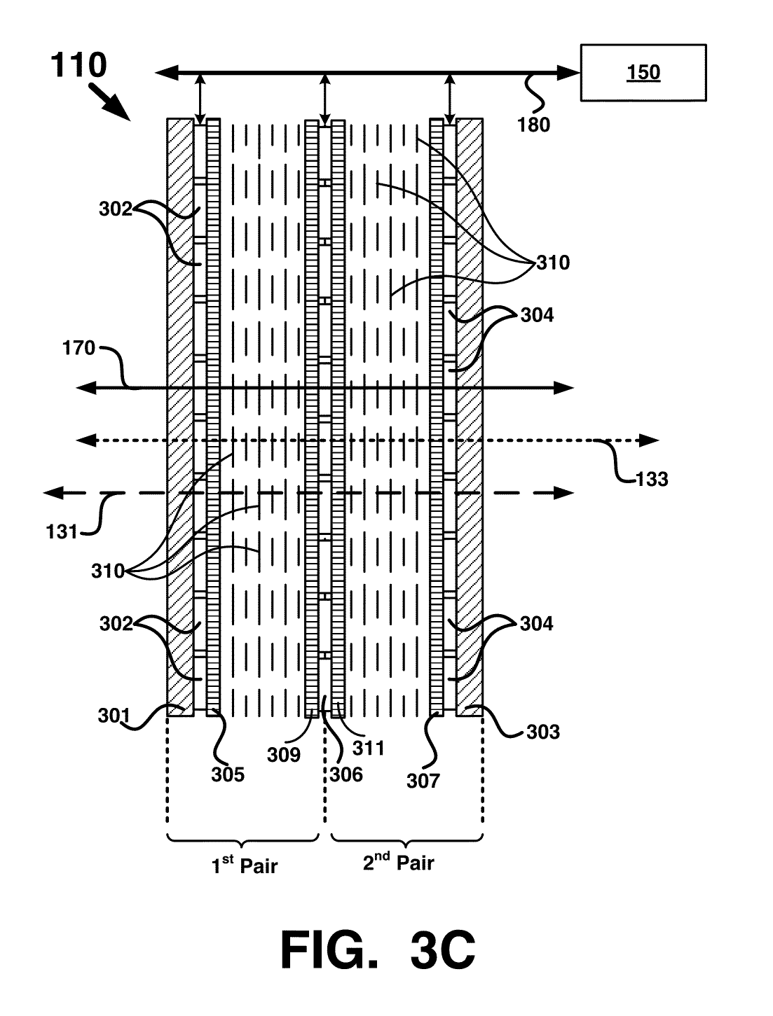

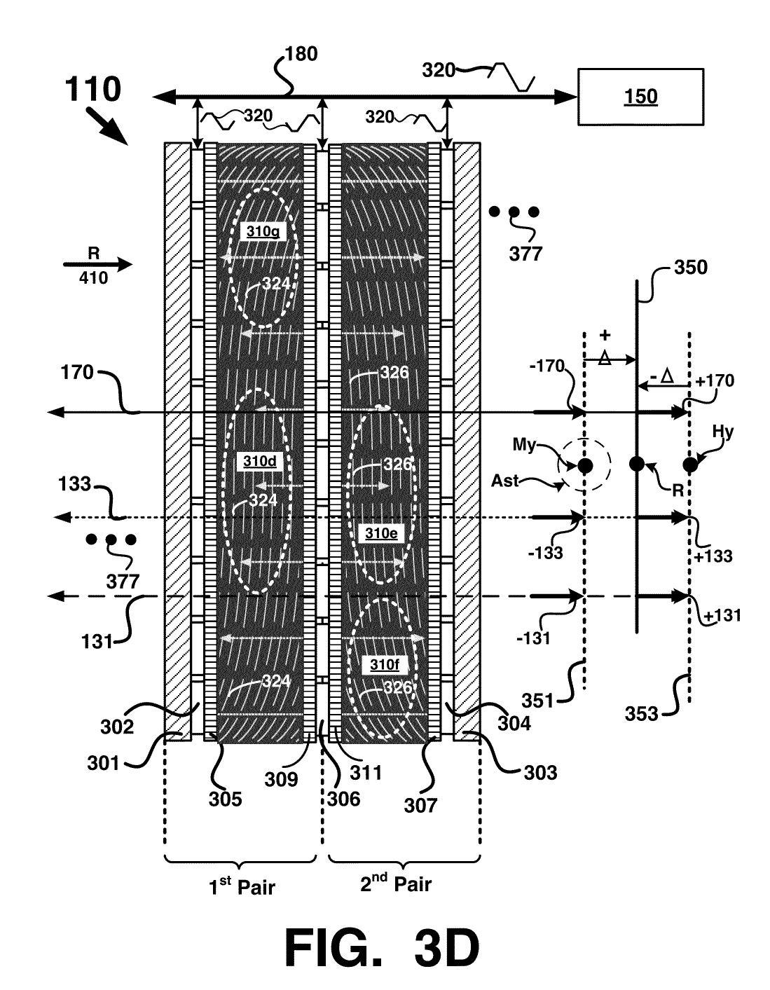

FIG. 3A depicts a cross-sectional view of one example of adaptive optics 110 coupled with a control system (e.g., display system 150). Hereinafter, systems, components, and the like associated with the right or left eyes (101, 103) may not include the “a” or “b” as part of their reference numerals. Adaptive optics 110 may comprise a liquid crystal display (LCD) that is optically transparent to light from images 170, 131, and 133. Adaptive optics 110 may include optically transparent glass substrates (301, 303), a plurality of optically transparent electrodes (302, 304) positioned between electrically insulating substrates (305, 307) and glass substrates (301, 303). Control signals 180 may be electrically coupled with optically transparent electrodes (302, 304) and display system 150. The optically transparent glass substrates (301, 303) may comprise electrically insulating and optically transparent substrates; therefore, substrates 301, 303, 305 and 307 may comprise electrically insulating substrates that are optically transparent and include the plurality of the optically transparent electrodes (302, 304) sandwiched between a pair of the electrically insulating and optically transparent substrates.

Adaptive optics 110 may include liquid crystals 310 disposed between electrically insulating substrates (305, 307) and operative to change alignment or orientation in response to an electric field generated by application of a potential difference across one or more of the optically transparent electrodes (302, 304). Adaptive optics 110 may comprise an imageless liquid crystal display in which light (170, 131, 133) passing through adaptive optics 110 and incident on the retina of eyes (101 and/or 103) is not perceived by a user as a visually discernible displayed image created by the orientation of the liquid crystals, but may instead be visually perceived as an image (e.g., an ambient image and/or projected image) that may be in focus, out of focus, or blurry, by operation of the liquid crystals affecting an index of refraction of the adaptive optics 110, for example.

Turning now to FIG. 3B where a cross-sectional view of one example of a control system 150 operative to modify an index of refraction of adaptive optics 110 of adaptive optics 110 is depicted. Here, reflected image 133 (e.g., reflected from retina R) is sensed by an image capture system in display system 150, is processed, and control signals 320 are generated by display system 150. The control signals 320 are coupled with a plurality of the transparent electrodes (302, 304) in one or more portions of adaptive optics 110 to cause the liquid crystals 310 in the one or more portions to alter their alignment (e.g., relative to the incident light 170, 131, 133) in response to electric fields 322 generated by a potential difference applied across electrodes (302, 304). For example, liquid crystals 310 disposed in a first portion of adaptive optics 110, denoted as 310a, may have their orientation slightly altered by lower magnitude electric fields 322 in portion 310a; whereas, in portions 310b and 310c, higher magnitude electric fields 322 more drastically alters orientations of liquid crystals 310 in portions 310b and 310c. As a result, an index of refraction may be higher in the portions 310b and 310c and light (170, 131, 133) passing through the portions 310b and 310c of adaptive optics 110 may be bent or curved more than light (170, 131, 133) passing through the portion 310a.

In FIG. 3B, a focal plane 350 may represent a retinal surface of an eye in optical communication with adaptive optics 110 and associated delivery optics 120 (not shown). Here, an arrow R 410 points in a direction of the retina R relative to the adaptive optics 110. Ideally, a person with perfect 20/20 vision would have all light in the visible spectrum focused on the focal plane 350 so that the light (170, 131, 133) incident on retina R produces a clear and focused image as perceived by the optical system and brain of a user. Accordingly, in an ideal eye, projected image 131, reflected image 133, ambient image 170, are all focused at the focal plane 350 of the retinal surfaces of the eye. However, any population of users may have nearsighted and farsighted users for who the light from images (170, 131, 133) will not converge into focus at the focal plane 350 of the retinal surfaces of the eye. For example, instead of focusing at the point R on focal plane 350, where R represent the retinal surfaces of the eye, a nearsighted user (e.g., myopia—My) will have light from images (170, 131, 133) converge in front of point R along a myopia plane 351 as denoted by images (−170, −131, −133). Blurriness of vision (e.g., astigmatism) may be denoted as a circle Ast that surrounds the point My on plane 351 and that circle may represent distortions caused by defects in the cornea, for example, that result in various degrees of fuzziness in images converging just before My or just behind My. Correction of the myopia and/or astigmatism may require a positive shift in position towards point R on plane 350 as denoted by +Δ. As another example, in contrast to the nearsighted example above, in a farsighted case, instead of focusing at the point R on focal plane 350, a farsighted user (e.g., hyperopia—Hy) will have light from images (170, 131, 133) converge behind point R along a hyperopia plane 353 as denoted by images (+170, +131, +133). Correction of the hyperopia may require a negative shift in position back towards the point R on plane 350 as denoted by −Δ. Adaptive optics 110 may effectuate the correction of focal point of images (−170, −131, −133, +170, +131, +133) by changing index of refraction in one or more portions of the liquid crystals 310 as described above. The change in index of refraction may cause light (170, 131, 133) to curve and converge at R instead of in front of or behind R if not corrected. Signals 320 applied by display system 150 may be selectively applied to specific electrodes (302, 304) to generate electric fields 322 in specific portions of adaptive optics 110. Electric fields generated in adaptive optics 110 may have different magnitudes and may have different directional vectors (e.g., a direction from electrode 302 to 304 or from 304 to 302). Signals 320 may have different magnitudes (e.g., in voltage or current) and polarities; therefore, signals 320 are not necessarily identical to one another and there may be variations in signal parameters among the signals 320. Actual waveform shapes for the signals 320 are not limited to the examples depicted herein. Signals 320 may be AC, DC or both.

FIG. 3C depicts a cross-sectional view of another example of adaptive optics 110 coupled with a control system 150. Here, adaptive optics 110 includes a plurality of stacked layers of liquid crystals 310 disposed between transparent electrodes (302, 306) and (304, 306). Electrically insulating substrates 309 and 311 define a region to the left and right where the liquid crystals 310 may be manipulated by electric fields to alter index of refraction in one or more specific portions of 110. There may be more stacked layers in adaptive optics 110 than depicted in FIG. 3C as denoted by 377. Stacked layers may share transparent electrodes (e.g., 302 and 304 sharing 306) or each layer may include electrodes that are not shared with electrodes in adjacent layers (not shown) such that the electrodes in adjacent layers are electrically isolated from one another. The optically transparent glass substrates (301, 303) may comprise electrically insulating and optically transparent substrates; therefore, substrates 301, 303, 305, 307, 309 and 311 may comprise electrically insulating substrates that are optically transparent and include the plurality of the optically transparent electrodes (302, 304, 306) sandwiched between a pair of the electrically insulating and optically transparent substrates.

In FIGS. 3C and 3D a layer of the liquid crystals 310 may be disposed between a first pair of adjacent electrically insulating and optically transparent substrates (e.g., 301, 305 and 309, 311) and another layer of the liquid crystals 310 may be disposed between a second pair of adjacent electrically insulating and optically transparent substrates (e.g., 309, 311 and 307, 303). Each pair of electrically insulating and optically transparent substrates sandwiches a plurality of the optically transparent electrodes (302, 304, 306). For example, 301 and 305 sandwich electrodes 302, 309 and 311 sandwich electrodes 306, and 307 and 303 sandwich electrodes 304. Additional layers of liquid crystals 310 may be included as denoted by 377.

Referring now to FIG. 3D where a cross-sectional view of another example of a control system 150 modifying index of refraction of adaptive optics 110 is depicted. Here, control signals 320 generate a plurality of portions 310d-310g that may be used to effectuate a change in the index of refraction as described above. Liquid crystals denoted as 324 in one layer and 326 in the other layer may be rotated according to the electric field present in the portions the liquid crystals (324, 326) are disposed in. Using multiple layers may be operative to provide greater control and/or finer gradations of change in index of refraction. For example, light converging in front of retina R around a myopia point My denoted as Ast for astigmatism may be reduced or eliminated by display system 150 applying signals 320 to specific electrodes (302, 304, 306) in the various layers to alter index of refraction to focus incident light on the point R instead of the Ast region around the point My. An amount of liquid crystals 324 and/or 326 in the different layer may be the same or different and may have different layer thickness. The number of electrodes may also differ between the multiple layers.

In the examples depicted in FIGS. 3A-3D, the materials that may be used for of adaptive optics 110 may include but are not limited to those that may be used in a variety of different LCD technologies and custom designed LCD panels, optics, or displays. Although not depicted in FIGS. 3A-3D, adaptive optics 110 may include additional thin film layers, materials, and structures such as polarizing films, thin-film-transistors (TFT), flexible polymers and plastics for a flexible adaptive optics 110, smectic, nemantic, isotropic, twisted STN, twisted cholesteric and other types of liquid crystals, just to name a few, for example.

In the examples depicted in FIGS. 3A-3D, adaptive optics 110 may comprise a non-linear optical lens having a varying index of refraction that is controllable by signals 320 (e.g., by a varying voltage applied to electrodes 302, 304, 306). Although flexible materials may be used for the adaptive optics 110, the varying index of refraction may be accomplished without curved optical surfaces or structures and the layers depicted may be planar or substantially planar layers. Planar surfaces may be advantageous for mounting or optically coupling the adaptive optics 110 with other optical structures such as the delivery optics 120. A planar surface of the delivery optics 120 may be coupled with a planar surface of the adaptive optics 110 using a press fit, adhesives, fusing, fasteners, or the like. Adaptive optics 110 may comprise a gradient-index of refraction (GRIN) lens. The GRIN lens may have flat or planar surfaces, arcuate surfaces or both. Planar surfaces may be advantageous for similar reasons as described above. Electrodes depicted in FIGS. 3A-3D may be addressed by control system 150 down to a granularity of a single electrode (e.g., a single pixel in an array of pixels) or a single pair of electrodes across which a potential difference may be applied to generate an electric field between the pair of electrodes. The electrodes may be disposed in an orderly configuration such as an array having rows and columns.

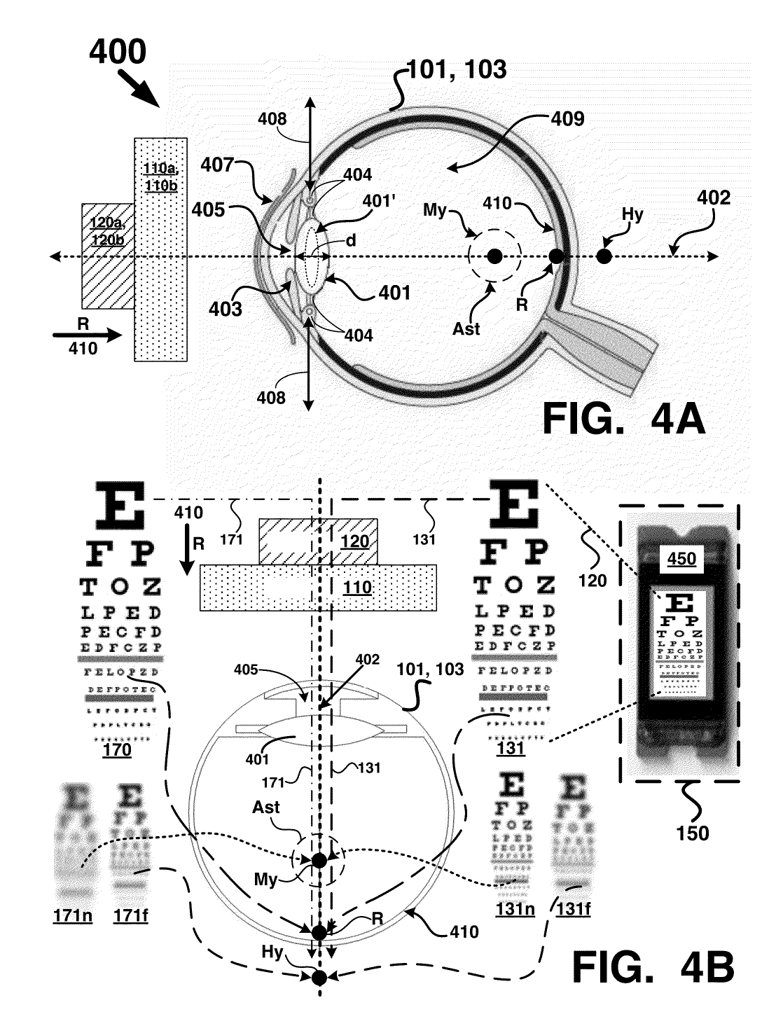

Now directing attention to FIG. 4A where a cross-sectional view of one example of an eye (101, 103) in optical communication with delivery optics (120a, 120b) and adaptive optics (110a, 110b) is depicted. Components of eye (101, 103) include: retina 410 where R depicts one of a plurality of points on retina 410 that ideally, light entering the eye converges at as a focal point; cornea 407; pupil 405; iris 403; lens 401 which may change dimensions d to focus light as denoted by smaller dimension 401′ in dashed line; ciliary muscles/suspensory ligaments 404 which relax and contract 408 to change dimension d to focus lens 401; and vitreous humor 409. An optical axis 402 is depicted symmetrically disposed through lens 401 and approximately aligned with delivery optics (120a, 120b) and adaptive optics (110a, 110b) for purposes of explanation only and is not a component of eye (101, 103). Points My and Hy denote spatial locations not on retina 410 where light passing through lens 401 may converge in instances of myopia and hyperopia. Dashed line Ast depicts an approximate region around myopia point My where light passing through lens 401 may converge in instances of astigmatism or other vision defects. Adaptive optics (110a, 110b) may be positioned to receive images (e.g., 131, 133) from delivery optics (120a, 120b) and ambient images (e.g., 171) and focus those images on surfaces of retina 410 (e.g., at point R or others on 410) as a focal point for clear visual perception by the user, for example. Adaptive optics (110a, 110b) may operate solely and/or in conjunction with systems of eye (101, 103) to alter focal point and/or index of refraction to cause light from (171, 131, 133) to focus at or approximately on retina 410 (e.g., at point R or others on 410).

For example, an image projected by display system 150 and optically coupled with eye (101, 103) via delivery optics (120a, 120b) sans the adaptive optics (110a, 110b) may converge in front of retina 410 at or around point My or behind retina 410 at or around point Hy, resulting in the projected image (e.g., 131) being out of focus as perceived by a user. The adaptive optics (110a, 110b) may be positioned relative to optical inputs to eye (101, 103) (e.g., from display system 150 and ambient 171) to bring images from those optical inputs into focus on retina 410 as denoted by the point R or other points on retina 410, such that images from those optical inputs appear to the user as being in focus.

FIG. 4B depicts a cross-sectional view of one example of direct 171 and projected images 131 and 133 presented to an eye (101, 103) in optical communication with delivery optics (120a, 120b) and adaptive optics (110a, 110b). For purposes of explanation, assume for example that an eye (101 and/or 103) of a user is viewing an eye chart 170 that is hung on a wall in room and display system 150 is projecting an image of the wall chart as image 131 that is optically coupled from a display 450 to the eye using the delivery system 120. The actual physical wall chart 170 comprises the light in ambient 171 that enters the eye. Ideally, as perceived by the user, wall chart image 170 from the ambient 171 and projected wall chart image 131 should both be in focus and converge at retina 410. However, if the user is near or far sighted, then one or both of the images 171 and/or 131 may appear to be out of focus.

As one example, image 170 from ambient 171 may be in focus at R; whereas, projected image 131n may be out of focus at My due to nearsightedness of the user, or image 131f may be out of focus at Hy due to farsightedness of the user. As another example, projected image 131 from display system 150 may be in focus at R; whereas, ambient image 171n may be out of focus at My due to nearsightedness of the user, or ambient image 171f may be out of focus at Hy due to farsightedness of the user. Eye 101 may be stronger or weaker than eye 103 and the in focus images at R and out of focus images at My or Hy may be different for each eye 101 or 103.

Adaptive optics 110 is operative to bring the ambient 171 and projected 131 images into focus at R for each eye (assuming each eye requires correction) so that both images appear sharp and well defined. Images 171, 131, 133 may be optically processed by adaptive optics 110 prior to entering the lens 401 of the eye (101, 103). Although an eye chart was used in the above example, the images in 171 and 131 may be different; however, adaptive optics 110 may be operative to bring different images into focus at R. For example, ambient 171 may be a street the user is walking along and display 450 in display system 150 may be projecting a GPS or location based map image 131. The image 131 may visually overlay the ambient 171, but both may be in focus from the point of view of the user who visually perceives the images 171 and 131.

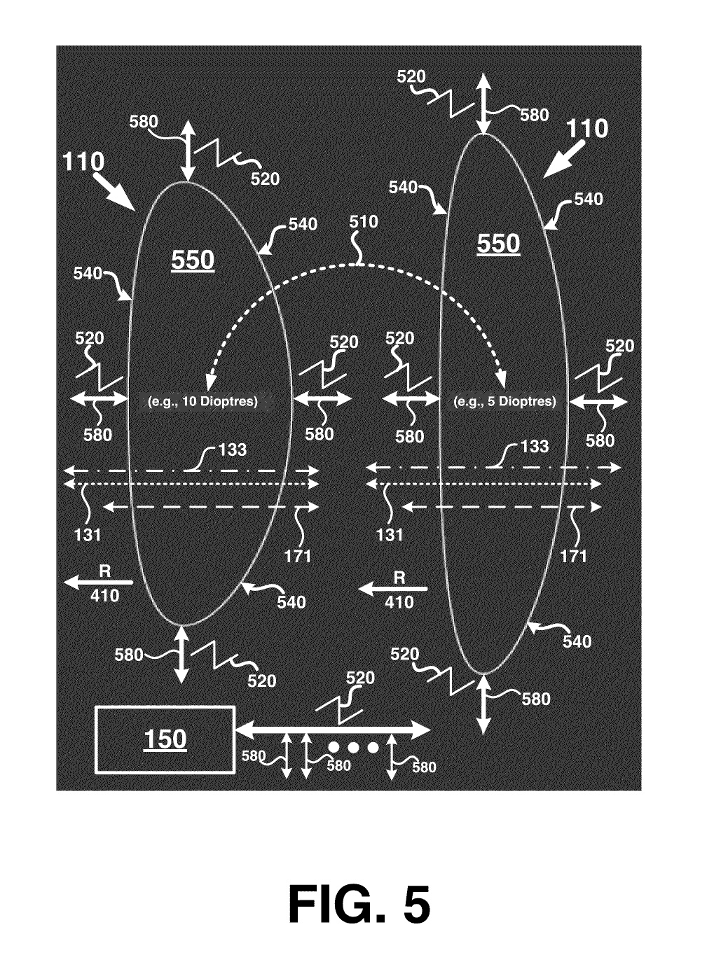

Turning now to FIG. 5 where a cross-sectional view of yet another example of adaptive optics 110 coupled with a control system 150 operative to modify an index of refraction of the adaptive optics 110 is depicted. Here an arrow for R 410 depicts a direction towards the retina 410 of an eye (not shown) positioned behind the adaptive optics 110. Delivery optics 120 are not depicted; however, for purposes of explanation, assume light 171, 131, 133 may pass through the delivery optics 120 and then through adaptive optics 110 in the direction of R 410. In FIG. 5, adaptive optics 110 may comprise a variable-focus lens having an optically clear body 540 (e.g., a flexible sealed volume) with an interior portion that is filled with an optically clear fluid 550. Actuators 580 may be coupled with portions of the optically clear body 540 and may be operative to compress, stretch pull or otherwise apply force to the optically clear body 540 to change optical properties of the adaptive optics 110 including its index of refraction. For example, actuators 580 may be electrically coupled with signals 520 which may be the same or different among the actuators 580 and responsive to the signals 520 each actuator receiving the signal 520 may apply force to the optically clear body 540 that collectively may reversibly change 510 an optical property such as refractive power of the adaptive optics 110 from one value to another value or vice-versa. For example, a first application of the signals 520 to adaptive optics 110 may change the refractive power from about 5 diopters as depicted on the right side of FIG. 5 to about 10 diopters as depicted on the left side of FIG. 5. As another example, a second application of the signals 520 to adaptive optics 110 may change the refractive power from the 10 diopters on the left, back to the 5 diopters on the right.

Actuators 580 may include but are not limited to piezoelectric actuators, a bending or flexing piezoelectric actuator, MEMs actuators, electromagnetic actuators, a linear motor, a stepping motor, a voice coil motor, a voice coil actuator, solenoid actuators, artificial muscle actuators, and transparent artificial muscle actuators, just to name a few. The transparent artificial muscle actuators may be optically transparent. Depending on the type(s) of actuators used, control system 150 may drive current, voltage or both via signals 520 to selectively effectuate activation of the actuators 580. One portion of actuator 580 may be coupled with the body 540 and another portion may be coupled with a portion of chassis 199 (e.g., an eyeglass rim). All or only a portion of the actuators 580 may be activated to change the optical property of the adaptive optics 110. Suitable optically transparent materials for body 540 include but are not limited to silicone, rubber, polymers, and synthetic rubbers, for example. Materials for fluid 550 may include but are not limited to refractive liquids, oil, synthetic oil, and water, for example. Here, changes in dimension and/or profile of the adaptive optics 110 may be operative to change index of refraction so that light from images 171, 133, 131 converges at the retina 410 as a focal point as described above.

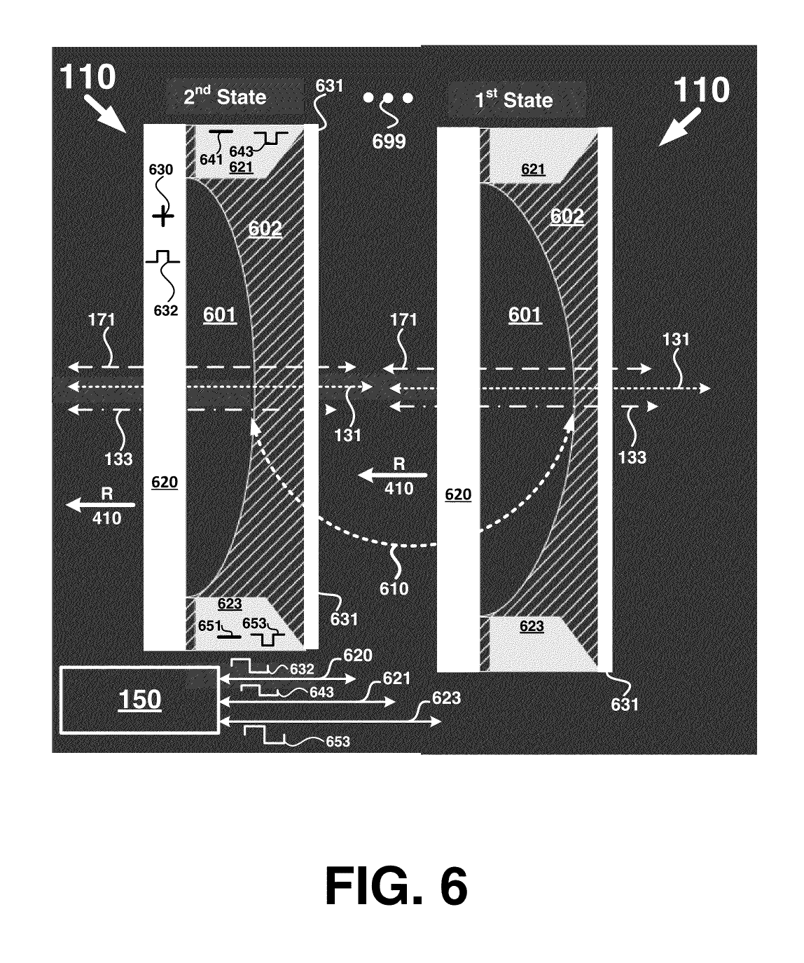

Reference is now made to FIG. 6 where a cross-sectional view of still another example of adaptive optics 110 coupled with a control system 150 operative to modify an index of refraction of the adaptive optics 110 is depicted. Adaptive optics 110 may comprise a sealed structure (e.g., a sealed optic) including a first liquid 601 and a second liquid 602 that are both optically transparent and preferably free of defects such as bubbles and/or particles, a first optically transparent electrode 620, electrodes 621 and 623 which may or may not be optically transparent, and an optically transparent window 631. The electrodes 621 and 623 may be in contact with the first liquid 601, the second liquid 602 or both. Light 131, 133, 171 passes through the aforementioned optically transparent materials in a direction towards retina as denoted by arrow R 410 and some of that light may be reflected back in a direction opposite to R 410. Liquid 601 may comprise a fluid such as water including a compound making it electrically conductive and liquid 602 may comprise an oil or dielectric oil, for example. Window 631 may be coated or covered with a layer of thin and electronically insulating hydrophobic material that is in contact with the second fluid 602.

Control system 150 may apply voltages to electrodes 601, 621 and 623 that are operative to cause the first fluid to reversibly change 610 shape and by so doing change an index of refraction of adaptive optics 110. As a result of the change in shape a focal length of adaptive optics 110 may be changed and the light 131, 133, 171 may be caused to focus at the retina 410 as its focal point. As one example, in a first state no voltage or a voltage of the same polarity may be applied to 601, 621 and 623 so that there is a potential difference of zero volts and no electric fields are generated. In a second state, control system may apply a negative voltage 641 and 651 to electrodes 621 and 623, respectively, and a positive voltage 630 to electrode 620 causing the first and second liquids to change shape from the shape on the right side of FIG. 6 to the shape on the left side of FIG. 6. The shape change is reversible 610 and the applied voltages may be removed from the electrodes and the first and second liquids 601 and 602 may revert back to the first state upon removal of those voltages as depicted on the right side of FIG. 6. The applied voltages may be in a range from about 1.5 volts to about 80 volts, for example, and those voltages may be application dependent and may be determined in part by the types of liquids used for the first and second liquids 601 and 602. Control system 150 may apply the voltages as DC voltages, AC voltages or both. AC voltages may be applied using a variety of waveform shapes, duty cycles, pulse widths, voltage magnitudes, and voltage polarities. For example, voltage 630 may be applied as a positive pulse 632 and voltages 641 and/or 651 may be applied as negative pulses 643 and/or 653, respectively. Voltages 641 and 651 may or may not have the same magnitude, polarity, or waveform shapes. Adaptive optics 110 may be used for auto-focus of the images in light 131, 133 and 171 and may also be used for optical image stabilization.

There may be a plurality of states for adaptive optics 110 other than the first and second states as denoted by 699. Changes in shape of the two liquids may be cycled in times as short as a few milliseconds and several tens of millions of cycles may be initiated without degradation in optical performance. Refractive power may be in a range from about 1 diopter to about 45 diopters, for example. Power consumption of electronics used to drive the adaptive optics 110 (e.g., in control system 150 or other circuitry and/or software) in device 100 may be in a range from about 5 mW to about 50 mW, for example. Optically transparent materials for electrode 620 and window 631 may be made from suitable glasses, plastics, polymers or the like and may be made from rugged and/or impact resistant materials such as Sapphire based glass or Corning® Gorilla® Glass, thus making the adaptive optics 110 resistant to shock from impacts or dropping of device 100, for example.

The adaptive optics 110 (e.g., 110a and 110b of FIGS. 1A and 1D) depicted in FIGS. 3A-3D, 5 and 6 may be used individually or in combination with one another (e.g., ganged to gather) to change index of refraction (e.g., change focal length) of a system that includes the adaptive optics, such as wearable device 100. For example, the adaptive optics 110 of FIGS. 3D and 6 may have their respective optical axes aligned (e.g., along axis 402 of FIGS. 4A-4B) and be used in combination or individually to correct index of refraction by increasing or decreasing their respective refractive powers (e.g., in diopters). As one example, the multi-layer liquid crystal adaptive optics 110 of FIG. 3D may adjust its refractive power in a range of about −8 diopters to about +7 diopters and electro wetting adaptive optics of FIG. 6 may adjust its refractive power in a range of about −2 diopters to about +3 diopters to fine tune the index of refraction adjustments needed to correct vision anomalies.

Adaptive optics 110a and 110b of FIGS. 1A and 1D may individually adjust their respective index of refractions as described above to change focal length or other optical property to bring images (e.g., light 131, 133, 171) into focus at or approximately at the retinas 410 of the right eye 101, the left eye 103 or both. Each adaptive optics 110 may have its own dedicated control system 150 or a single control system 150 may control a plurality of adaptive optics 110 (e.g., may control 110a and 110b, or others). Adaptive optics 110a and 110b of FIGS. 1A and 1D may individually adjust their respective index of refractions as described above to compensate for vision defects and/or disease of the right and left eyes 101 and 103, respectively.

Attention is now directed to FIG. 7 where a block diagram 700 of one example of a display system 150 optically coupled with an eye through delivery optics 120 is depicted. Components of display system 150 may include but are not limited to an image projector 720, an image capture device 730, an ambient light sensor 790, optics 777, and a communications interface 770. A processor 701 may be included in display system 150 or may be included in wearable device 100 and electrically coupled with components of display system 150. If there are multiple display systems 150 (e.g., 150a and 150b), then each display system may include its own processor 701, only one of the display systems may include the processor 701, or the wearable device 100 may include the processor 701 which is electrically coupled with the components of each display system (e.g., 150a and 150b). Processor 701 may include data storage DS 703 (e.g., Flash memory, embedded memory), algorithms fixed in a non-transitory computer readable medium (e.g., DS 703), and configuration data CFG 707 that may be used to configure operations, functionality, etc. of display system 150. Communications interface 770 may be coupled 771 with processor 701 and may include I/O circuitry 776 for wired communications 775 and/or wireless communications 772 using one or more wireless communications protocols over one or more radio frequencies (RF) 773. Images 131p to be projected by image projector 720 may be wirelessly communicated 773 to display system 150 by an external wireless client device (not shown), such as a smartphone, tablet, pad, or wireless network, for example. Optics 777 may be any form of optical system or components that may be operable for coupling light 131 from projector 720 with delivery optics 120 and for coupling light 133 from delivery optics 120 with image capture device 730. In the non-limiting example depicted in FIG. 7, optics 777 may comprise a beam splitter prism operative to optically reflect incident light 133 from projector 720 to optics 778 (e.g., a mirror) in delivery system 120 and also operative to optically couple reflected light 133 from delivery system 120 with image capture system 730. Delivery optics 120 may include optics 779 operative to couple light 131 into eye (101, 103) via adaptive optics 110. In the non-limiting example depicted in FIG. 7, optics 779 may comprise a beam splitter prism operative to optically reflect light 131 from projector 720 into the adaptive optics 110 and into eye (101, 103), to optically couple reflected light 133 with optics 778 and 777, and to optically couple ambient light 171 into adaptive optics 110 and into eye (101, 103). Ideally, all of the light (e.g., 131, 171) entering eye (101, 103) would focus at the retina 410 as its focal point (e.g., point R and others on retina 410). However, due to vision anomalies, such as astigmatism, glaucoma, nearsightedness, and farsightedness, for example, the light may be spatially displaced by some measurable distance from point R as denoted by points Dp that may be positioned above and/or below point R. As described above, display system 150 may apply signals 780 to adaptive optics 110 to correct or reduce one or more of the vision anomalies as will be described in greater detail below. Circuitry in processor 701 or in electrical communication with processor 701 may apply signals 780 to the adaptive optics 110 described in FIGS. 3A-3D, 5 and 6 to reversibly change a refractive power (e.g., index of refraction and/or focal length) of the adaptive optics 110. In FIGS. 3A-3D the circuitry may selectively address one or more of the plurality of optically transparent electrodes (302, 304, 306) to apply voltage potentials to for generation of the electric fields in the one or more layers of liquid crystals 310 to change a refractive power of the adaptive optics 110. The electric fields so generated may have different magnitudes and directions in different portions and/or regions of the layers of liquid crystals 310.

A variety of display systems and their associated optical component, light engines, backlights, polarizers, prisms, total-internal-reflection (TIR) prisms, and display engines (e.g., DLP, DMD, LCD, LCoS, OLED, transmissive, reflective, active matrix, passive matrix, etc.) may be used in projector 720 and the example depicted in FIG. 7 is non-limiting. Projector 720 may be electrically coupled (745, 747) with processor 701 and may include an image display 721 for displaying an image 131p from image data electrically coupled 745 with driver circuitry of display 712. Image 131p may comprise an image of a model pattern used for correcting the aforementioned vision anomalies or may be image data, such as a pie chart 798 depicted at the bottom of FIG. 7. Projector 720 may further include a light source 723 (e.g., a backlight) that is electrically coupled 747 with processor 701 which may drive signals that strobe or otherwise activate one or more different color light emitting devices (e.g., Red, Green, Blue, and optionally Ir LED's and/or Lasers) in light source 723. The light emitting devices (e.g., RGB LED's and/or Lasers) may be arranged in an array structure. An infrared Ir light emitting device may be included in light source 723 (e.g., in the array structure) or may be positioned separately from the Red, Green, Blue light sources. In some examples, an opto-electronic device such as a LED or laser may include separate semiconductor die for each of the Red, Green, Blue and Ir light sources. Each device may have a separate anode that is electrically driven to activate the device and the devices may share a common cathode, or vice-versa. Other components that may be included in light source 723, such as a homogenizer, micro-lens arrays, polarizers, diffusers, and the like are not depicted and may be application dependent. In some examples, light from light source 723 may be optically coupled with image display 721 using a TIR prism (not shown) and a light output face of the TIR prism (e.g., where light 131 exits the TIR prism) may be optically coupled with optics 777 (e.g., a beam splitter). The IR light source may include one or more of the above mentioned components that may be included in light source 723 and/or may include optical structure operative to create structured light for diagnosing problems in eyes (101, 103), such as a grating (e.g., a holographic grating or other types of gratings), for example. Light source 723 may comprise an integrated (e.g., monolithically integrated on a semiconductor substrate) optoelectronic device including RGBIr LED's or RGBIr lasers, for example. In other examples, a White light source such as a White LED or laser may be included in light source 723 as a discrete light emitting device or an integrated light emitting device that may be integrated with other light emitting devices that emit light of different wavelengths such as one or more of the Red, Green, Blue or Ir light sources, for example. In some examples, the White light source may be generated by a plurality of light sources having wavelengths that when optically combined generate the White light.

Image capture device 730 may include a solid-state imaging sensor 731 (e.g., CMOS image sensor or CCD image sensor) that may be included in a housing 733 with optics 736 to focus image 133 onto the image sensor 731, and image processing circuitry electrically coupled 745 with processor 701 and operative to communicate captured image data to the processor 701. As will be described below, an image 133i of the model pattern 131p (after being reflected off of retina 410) may be imaged onto image sensor 731 using the optical path depicted in FIG. 7 and displacements in that image when compared against an ideal model may be used to generate signals 780 operative to cause the adaptive optics 110 to change index of refraction (e.g., focal length) to correct or reduce the aforementioned vision anomalies. After correction by adaptive optics 110, image 799 (e.g., a statue) from ambient light 171 and an image 798 (e.g., a pie chart being projected by projector 720) should both be in focus at point R on retina 410 with little or no displacement Dp. Image capture device 730 and/or its image sensor 731 may be aligned on-axis as depicted in FIG. 7 or may be aligned off-axis. An off-axis alignment may be advantageous for detecting optical imperfection in the eyes (101, 103) by rendering the aforementioned structured light caused by those imperfections more obviously variant than may be the case for an on-axis alignment of 730 and/or 731. The off-axis alignment need not be a major misalignment and may comprise a slight displacement of 730 and/or 731 from the on-axis alignment position.

Display system 150 may also include an ambient light sensor 790 having a light sensing device 791 (e.g., a photo diode or other opto-electronic light sensing device) and associated circuitry electrically coupled 795 with processor 701 and operative to generate a signal on 795 (e.g., an analog and/or digital signal) indicative of ambient light 792 incident on sensing device 791. Iris 403 of eye (101, 103) may dilate or constrict pupil 405 in response to arousal in the sympathetic nervous system (SNS) and/or in response to ambient light conditions. Ambient light sensor 790 may be used to determine if ambient light conditions are too bright to accurately image retina 410 (e.g., because the pupil is constricted) using the pattern 131p projected by 720. Moreover, light other than light in the visible spectrum for human beings (e.g., infrared Ir from light source 723) may be used to prevent the pupil from constricting when the pattern 131p is being projected by 720. Another system in wired 775 and/or wireless 773 communications with device 100 or display system 150 may communicate data indicative of arousal state of the SNS and that data may be used to determine if the retina 410 may be imaged using reflected light 133 from projection of pattern 131p, for example. As one example, a data capable strapband, fitness monitor, smartwatch, or other wired and/or wireless client device may communicate (775, 773) sensor data from biometric sensors operative to sense arousal of the SNS (e.g., skin conductance, galvanic skin response—GSR, electromyography—EMG, etc.) and that sensor data may be used in a calculus (e.g., analysis by processor 701) for determining if conditions are conducive for reliable imaging of retina 410. In some examples, wearable device 100 and/or display system 150 includes an arousal sensor. Each display system (e.g., 150a, 150b) may include its own ambient light sensor 790, or a single ambient light sensor 790 may service more than one display system (e.g., 150a, 150b).

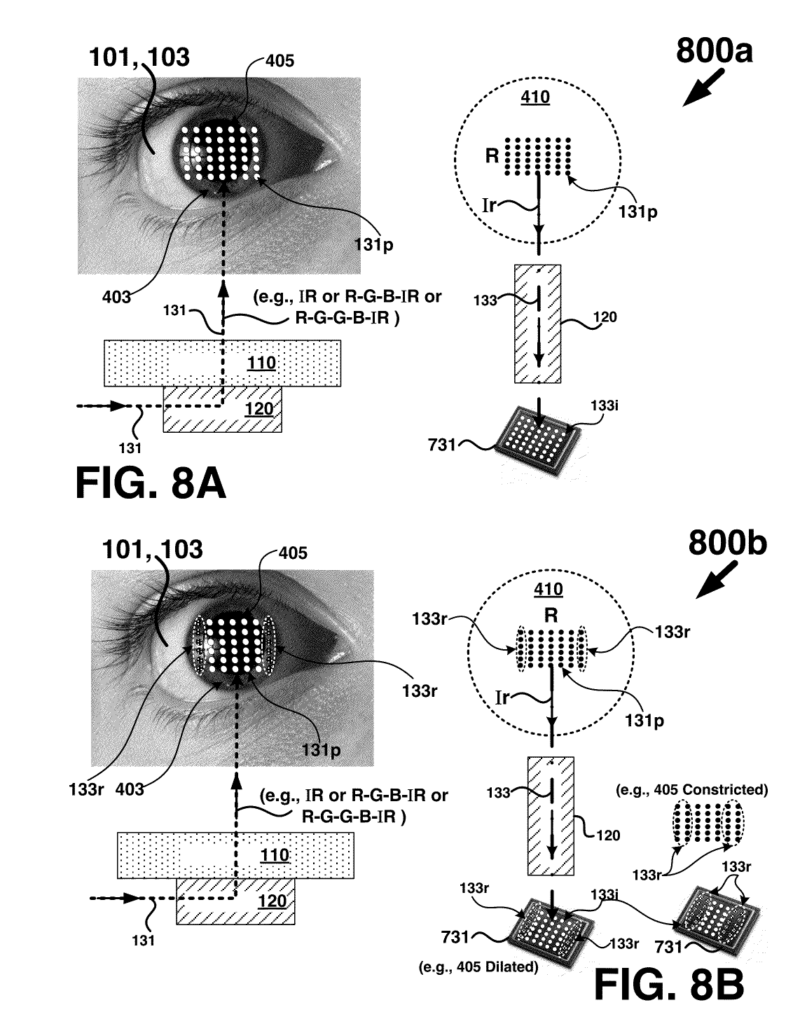

Moving on to FIG. 8A where one example of a model image projected into an eye (101, 103) by delivery system 120 is depicted. In FIGS. 8A and 8B, for purposes of explanation, optical components of delivery system 120 and adaptive optics 110 are not shown. Now, using delivery system 120 as described above in FIG. 7, light 131 from projector 720 is optically coupled with eye (101, 103) such that a model pattern 131p being displayed on 723 is projected into the eye (101, 103) as depicted by the white dots of the pattern 131p incident on the iris 403 and pupil 405. Light 131 may in some examples comprise infrared Ir from light source 723 or some other light source in display system 150, as described above. Infrared Ir light may be used instead of or in combination with one or more of the Red, Green and Blue light sources. The IR light may be used in lieu of the Red, Green and Blue light sources to prevent constriction of pupil 405 that may otherwise occur due to high ambient light conditions and/or a reaction by eye (101, 103) to the Red, Green and Blue light sources.

In some examples, the light sources may be strobed or otherwise activated and deactivated in some sequence that allows the pattern 131p to be projected without causing constriction of pupil 405, so that pupil 405 remains sufficiently dilated during the imaging process. For example, light source 723 may be controlled 747 by processor 701 and/or by its own circuitry to activate only the IR light source, to activate all light sources in a pattern such as strobing in a predetermined sequence, Red-Green-Blue-IR or Red-Green-Green-Blue-IR, for example. Pulse width modulation and current may be controlled to control duration of activation and light intensity, for example.

Now to the right of FIG. 8A, retina 410 is depicted in dashed line and the image of the model pattern 131p being projected on the retina 410 is depicted as being perfect, that is without any displacement Dp caused by vision anomalies that would require correction by adaptive optics 110. Some of the IR light in 131 that is incident on retina 410 is reflected back through 110 and 120 onto image sensor 731 of image capture device 730 (see FIG. 7) as light 133 that includes reflected image 133i depicted as incident on the sensor array of 731. Signals 735 generated by the image 133i may be processed by processor 701 to determine if the signals match or don't match those of the model pattern, which may be stored as a file, data structure, look-up table or some other data format in DS 703, for example. In FIG. 8A, the incident pattern image 131i does not include displacement Dp as mentioned above, and processor 701 may not generate signals 780 to adaptive optics 110 to correct vision anomalies by changing its index of refraction, for example.

Moving down to FIG. 8B where another example of a model image projected into an eye (101, 103) by delivery system 120 is depicted. In FIG. 8B, the description for FIG. 8A may still apply; however, one or more portions of the incident light in pattern 131p may be reflected off of the iris 403 with a different intensity than the pupil 405 as denoted by the dashed lines for reflected light 133r. Here, incident light from pattern 131p may enter into pupil 405 with little or no reflection; whereas, the iris 403 may block and/or reflect 133r those portions of the light 131p that are incident on the iris 403. The reflected light 133r from iris 403 and reflected light 133 from retina 410 may be optically coupled with image sensor 730 and may have different intensities and/or color components on the array of sensor 731 as depicted by dashed lines for 133r on the array. Signals 735 from the sensor 730 may be processed by processor 701 to determine that the signals 735 are indicative of the pupil 405 being sufficiently dilated for performing accurate imaging of retina 410. The determination of sufficient dilation may be made in conjunction with other signals, such as signal 795 from ambient light sensor 790, biometric signals from the aforementioned arousal sensors or both. In contrast, when the pupil 405 is constricted, then more of the pixels in array 731 may have different intensities and/or color components and processor 701 may process signals 735 to determine the pupil is not sufficiently dilated for accurate imaging of retina 410, for example. The determination of insufficient dilation may be made in conjunction with other signals, such as signal 795 from ambient light sensor 790, biometric signals from the aforementioned arousal sensors or both.

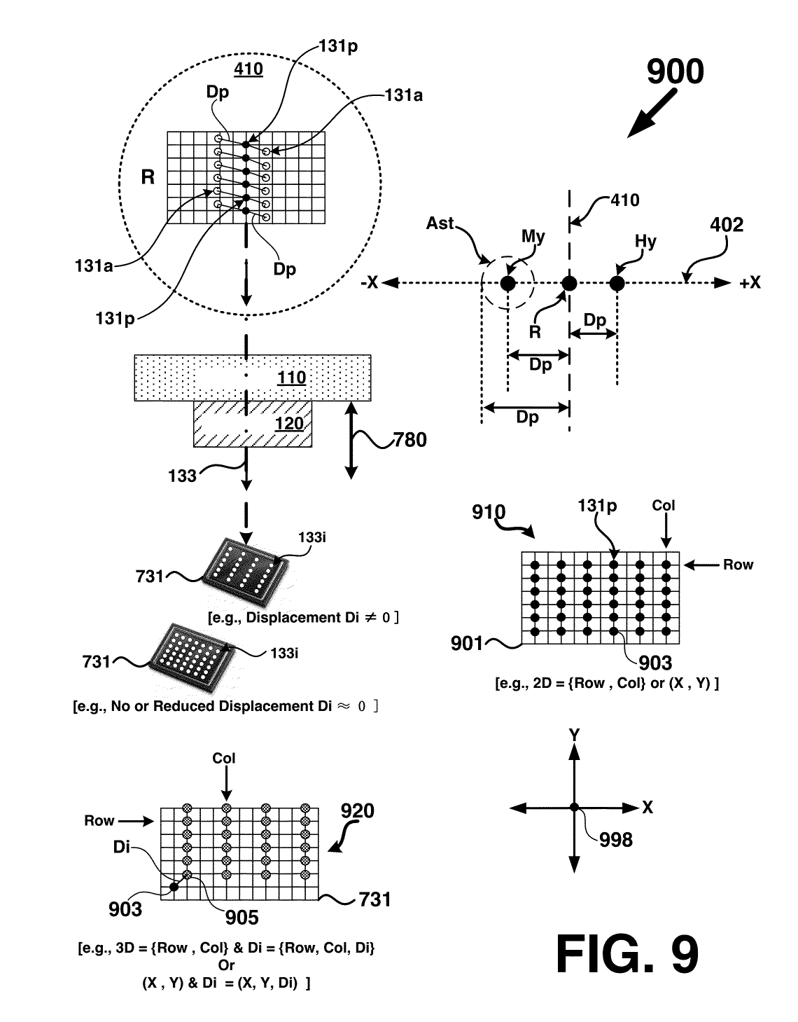

Referring now to FIG. 9 where one example of sensing image displacement Dp for vision correction using adaptive optics 110 is depicted. Now in FIG. 9, a model image pattern 910 that is free of distortion or displacement DP, may be stored in DS 703 or other location and may be driven via signals on 745 onto the image array 721 of projector 720 to be projected as the image 131p as depicted in FIG. 7. The model image pattern 910 includes dots 903 that for purposes of explanation are aligned at intersections of row and column lines. As described above, due to vision anomalies, defects in lens 401 or other, light in the image 131p that is incident on the retina 410 may be displaced and therefore not come into convergence or focus at point R on retina 410, but instead converges at points My, Hy or Ast. For purposes of explanation, the spatial distance of those points (My, Hy, Ast) from R is denoted as displacement distance Dp. Retina 410 is denoted in dashed line with one column of the model pattern image 131p depicted with its dots being on aligned on grid just as depicted in 910. However, the actual image 131a on the retina 410 is not perfect as in the model pattern image 131p and the dots for the actual image 131a and therefore its reflection incident on the image sensor 731 of the image capture device 730 exhibits displacement Dp as depicted by the dots in the actual image 131a that are spatially displace down and to the right of the perfect dots of the model pattern image 131p or up and to the left of the model pattern image 131p. In either case, image sensor 731 captures an imperfect image 920 that includes dots 905 that are spatially displaced on the image array of sensor 731 by some increment (e.g., pixel pitches) denoted as Di from where they ideally ought to be if there were no vision anomalies as denoted by 903 (e.g., the ideal location from 910). Therefore, the actual coordinate position of dot 905 is up one row and over one column from the ideal coordinate position of the dot 903 from model 910 as denoted by the line Di between those two dots. In the model image pattern 910 each dot 903 may be assigned a two-dimensional (2D) address or vector within the pattern 910, such as its row and column or its X and Y coordinate relative to some reference point in the pattern 910. In that the convergence of the light on retina 410 may spatially be in front of R (e.g., My or Ast) or behind R (e.g., Hy), the displacement Di of the dots 903 in the imperfect image 920 may be sensed by 731 and output as the signal 735 from image capture device 730.

Processor 701 may process the Di signal and combine it with the 2D address for the ideal dots 903 to calculate a three-dimensional (3D) address or vector for displaced images and the 3D address or vector may be used to generate the control signals 780 coupled with adaptive optics 110 to cause the adaptive optics 110 to adjust its index of refraction until Di on the image sensor 731 is zero or reduced by some predetermined value. Adjusting the index of refraction may cause the Dp for myopia My to retard in the +X direction on axis 402 back towards point R and may cause Di on image sensor 731 to be reduced. Similarly, adjusting the index of refraction may cause the Dp for hyperopia Hy to advance in the −X direction on axis 402 back towards point R and may also cause Di on image sensor 731 to be reduced. Processor 701 may use a feedback loop or other process to continually signal 780 the adaptive optics 110 to change index of refraction until Di is reduced or is zero. The feedback loop may include continuing to project the model image pattern 131p, calculating Di in the reflected image 133r, applying signals 780 to adjust index of refraction on 110, and repeating until Di is reduced or is zero. In some applications the 3D address may comprise the ideal 2D address given as (Row, Col) or (X, Y) and the displacement Di to determine the 3D address as (Row, Col, Di) or (X, Y, Di). In other applications, the displacement Di may comprise an address or coordinate of the displaced image such as (Di-X, Di-Y) or (Di-Row, Di-Col), for example and the 3D address may comprise the ideal and displace addresses or coordinates, such as (Row, Col); (Di-Row, Di-Col) or (X, Y); (Di-X, Di-Y), for example. X and Y coordinates may be determined relative to an X-Y axis 998 that may include an origin (e.g., (0,0)) assigned to some position in one or more of array 731, pattern 910 or pattern 920, for example. X-Y axis 998 may be a software construct used by algorithms (e.g., ALGO 705) embodied in a non-transitory computer readable medium executing on processor 701 and/or other compute engine, for example.

Optical structures in wearable device 100 may be designed and/or simulated using CAD and EDA software tools such as MATLAB®, SYNOPSYS® CODE V®, Mathematica®, open source design and simulation tools, just to name a few. Optical structures in wearable device 100 may include but are not limited to linear optics, non-linear optics, aspheric lens and/or optics, flexible optics, inflexible optics, color filtering optics, beam splitters, x-cubes, total-internal-reflection (TIR) prisms, mirrors, wave plates, lens arrays, homogenizers, solid state light emitting sources (e.g., color and/or monochrome LED's and/or lasers), backlight optics, and polarizing optics, just to name a few, for example.

CAD and EDA hardware design, simulation and verification tools such as those from SYNOPSYS®, or Cadence® may be used to design display driver circuitry in display system 150. One or more processor (e.g., μP, μC, DSP, or ASIC's) and/or electrical systems included in chassis 199 and/or display systems (150a, 150b) may be used to control various electrical functions, adaptive optics, and execute algorithms fixed in a non-transitory computer readable medium.

Although the foregoing examples have been described in some detail for purposes of clarity of understanding, the above-described inventive techniques are not limited to the details provided. There are many alternative ways of implementing the above-described techniques or the present application. Waveform shapes depicted herein are non-limiting examples depicted only for purpose of explanation and actual waveform shapes will be application dependent. The disclosed examples are illustrative and not restrictive.

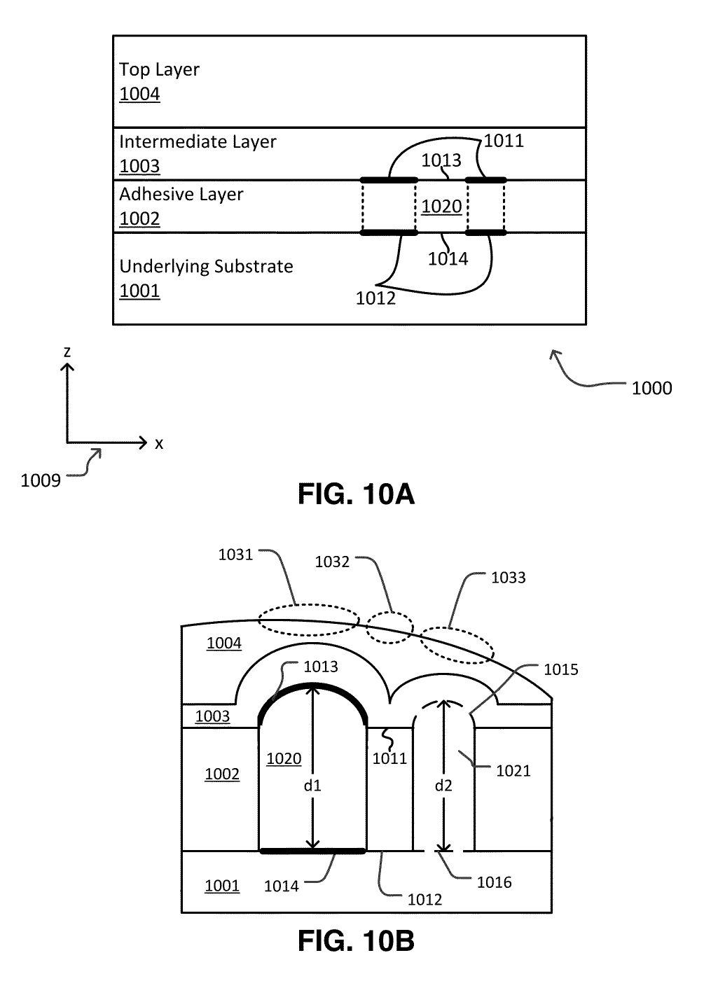

FIGS. 10A and 10B depict a cross-sectional view of an example of an adaptive optical material using one or more fluids, according to an embodiment. As shown, FIG. 10A depicts adaptive optical material 1000 having an underlying substrate 1001, adhesive layer 1002, intermediate layer 1003, and top layer 1004. FIG. 10A also depicts an x-z axis 1009, with +z indicating an upwards direction with respect to the figure. Adaptive optical material 1000 may be configured to focus collimated light rays substantially at a point (e.g., focal point, or point of focus), and to reversibly or adaptively change a distance between this point and adaptive optical material 1000 (e.g., focal length) using one or more fluids. In some examples, a point of focus may be behind underlying substrate 1001 (e.g., below adaptive optical material 1000, or in the −z direction from adaptive optical material 1000), may be in front of top layer 1004 (e.g., above adaptive optical material 1000, or in the +z direction from adaptive optical material 1000), or in another location. In some examples, adaptive optical 1000 may be configured to be worn, such that an eye is placed behind underlying substrate 1001 (e.g., in the −z direction from adaptive optical material 1000), and ambient light rays projecting an ambient image may enter through top layer 1004 and exit through underlying substrate 1001 to the eye. Adaptive optical material 1000 may include a multi-layer stack of transparent layers or materials, which may together form an optical lens. In some examples, layers or materials of adaptive optical material 1000 may have indices of refraction that are substantially equal. In some examples, intermediate layer 1003 may be a substantially flexible material such as a transparent polymer disposed between underlying substrate 1001 and top layer 1004. Intermediate layer 1003 may be configured to distend, expand, contract, or change shape when a pressure is applied in the z direction against intermediate layer 1003. The ability to expand with pressure may be characterized by a level of compliance (e.g., flexibility). For example, as pressure is applied in the +z direction, intermediate layer 1003 may expand in the +z direction. As pressure is decreased, intermediate layer 1003 may recoil and become substantially flat. Thus, a shape of intermediate layer 1003 may reversibly change based on an amount of pressure applied to intermediate layer 1003.

In some examples, top layer 1004 may also be a substantially flexible material. Top layer 1004 may be configured to expand or contract when a pressure is applied against top layer 1004. A shape of top layer 1004 may reversibly change based on an amount of pressure applied to top layer 1004. In some examples, a level of compliance of top layer 1004 may be less than a level of compliance of intermediate layer 1004. A material that is less compliant than another material may deform or expand less than the other material when a same pressure is applied to both materials. Therefore, given a same pressure, top layer 1004 may have a smaller change in curvature than intermediate layer 1004. In some examples, various pressures may be applied in the +z direction to portions of intermediate layer 1003, causing portions of intermediate layer 1003 to expand in the +z direction. These expanded portions may cause a surface of intermediate layer 1003 to be bumpy or uneven or to have high rates of change in curvature. These expanded portions may also assert a pressure in the +z direction against top layer 1004. Since top layer 1004 may be less compliant or flexible than intermediate layer 1003, top layer 1004 may deform less. Thus, a surface of top layer 1004 may be less uneven and more smooth.

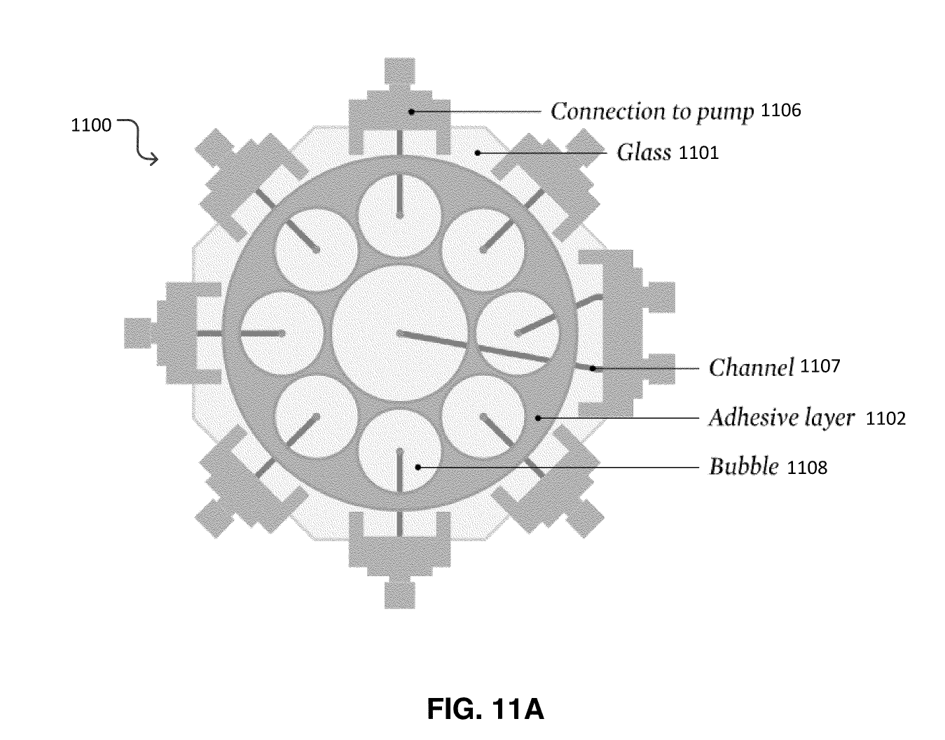

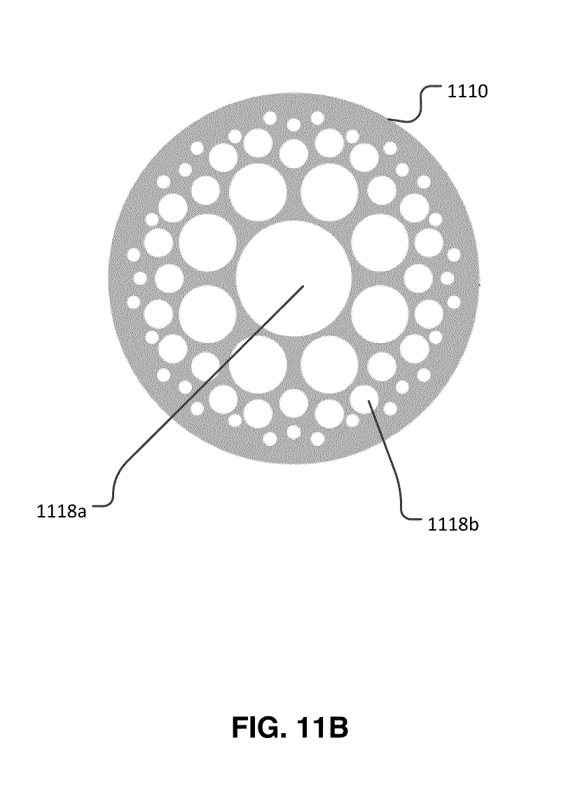

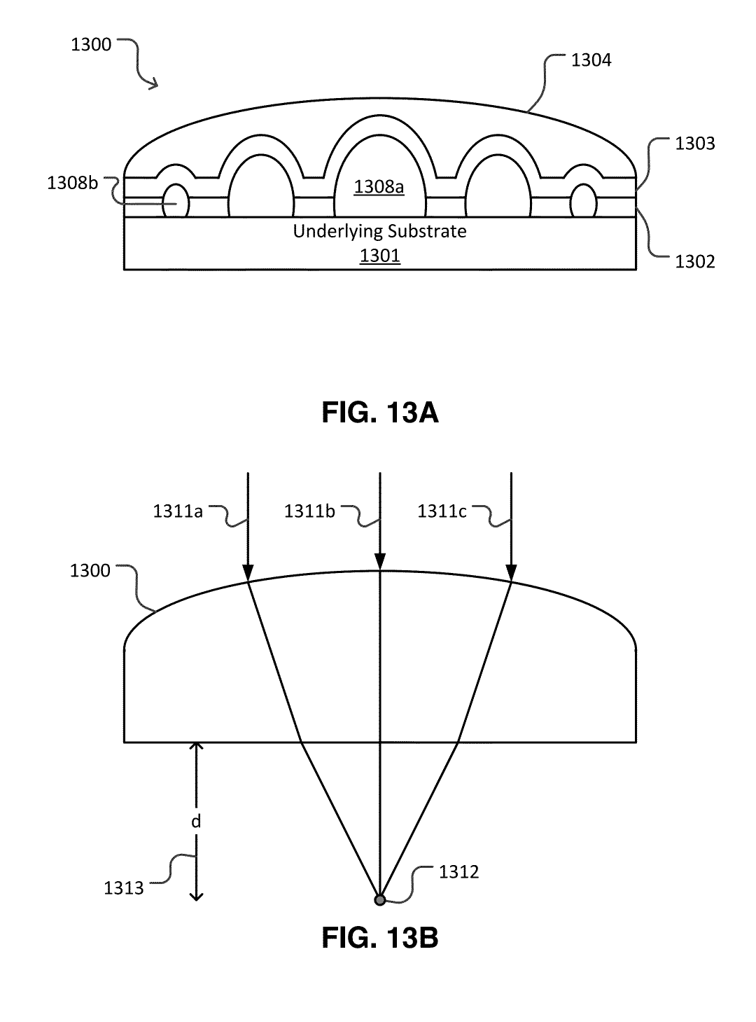

In some examples, adhesive layer 1002 may be disposed between intermediate layer 1003 and underlying substrate 1001 and may serve to adhere portions 1011 of intermediate layer 1003 to portions 1012 of underlying substrate 1001. Portions 1013 of intermediate layer 1003 that are not adhered to portions 1014 of underlying substrate 1001 may form one or more cells 1020 (used interchangeably herein with “bladders” or “bubbles”). Bladder 1020 may be configured to receive a fluid through a channel extending from adhesive layer 1002 or underlying substrate 1001. A bladder may change in volume or shape based on a volume of fluid received. As a bladder becomes thicker, the bladder may assert pressure against intermediate layer 1003, and portions 1013 of intermediate layer 1003 may expand. As portions 1013 of intermediate layer 1003 expands, portions of top layer 1004 may expand. A curvature or shape of top layer 1004, a curvature or shape of intermediate layer 1003, one or more volumes of bladders, and other parameters may change a focal length or other characteristic of adaptive optical material 1000. Bladders, or bubbles, are further discussed herein (e.g., FIGS. 11A, 11B, 13A, 13B, etc.).

In some examples, underlying substrate 1001 may be a substantially rigid material such as glass. In some examples, underlying substrate 1001 may have substantially flat and parallel top and bottom surfaces (as shown). In other examples, underlying substrate 1001 may be a different shape, for example, having one or more convex or concave surfaces. Still, a multi-layer stack of adaptive optical material 1000 may exclude one or more elements 1001-1004, and may include other types of layers or portions of layers, and the order of layers may be different.

As shown, FIG. 10B depicts underlying substrate 1001, adhesive layer 1002, intermediate layer 1003, and top layer 1004. FIG. 10B also depicts surface portions 1011, 1013-1016, and 1031-1033. In some examples, intermediate layer 1003 may be a flexible material disposed between underlying substrate 1001 and top layer 1004. Top layer 1004 may also be a flexible material. In some examples, a portion 1011 of intermediate layer 1003 may be fixed to a portion 1012 of underlying substrate 1001 by adhesive layer 1002. In other examples, portions 1013 and 1015 of intermediate layer 1003 may not be fixed to portions 1014 and 1016 of underlying substrate 1001 by adhesive layer 1002. For example, surface portions 1013 and 1014 form bladder 1020, and surface portions 1015 and 1016 form bladder 1021. In some examples, surface portions 1013 and 1015 be adjacent to, may face, or may be otherwise associated with intermediate layer 1013, while surface portions 1014 and 1016 may be adjacent to, may face, or may be otherwise associated with underlying substrate 1001. In some examples, as bladders 1020 and 1021 receive a fluid, a volume of bladders 1020 and 1021 increase, and surface portions 1013 and 1015 may expand. Surface portion 1011 may not expand since it is fixed by adhesive layer 1002. As surface portion 1013 expands, distance d1 between surface portion 1013 and surface portion 1014 may increase. Similarly, as surface portion 1015 expands, distance d2 between surface portion 1015 and surface portion 1016 may increase. Surface portions 1013 and 1015 may push against surface areas 1031-1033 of top layer 1004, and may cause surface areas 1031-1033 to expand.

As described above, intermediate layer 1003 and top layer 1004 may be flexible materials. A flexible material may be configured to bend, deform, expand, contract, or change shape based on a pressure applied against it. A level of compliance or flexibility may be a measure of how much a material deforms or changes shape based on a pressure. In some examples, a shape of a surface of top layer 1004 may follow a shape of a surface of intermediate layer 1003. For example, bladder 1020 may be larger than bladder 1021, and an expansion of surface area 1013 may be greater than that of surface area 1015. Thus an expansion of surface area 1031, which is facing or opposite to surface area 1013, may be greater than that of surface area 1015, which is facing or opposite to surface area 1015.

In some examples, top layer 1004 may be less compliant than intermediate layer 1004. Top layer 1004 may deform less than intermediate layer 1004 based on a substantially similar pressure applied to the layers. For example, a curvature of surface portions 1031 and 1033 may be less than that of surface portions 1013 and 1015. As another example, surface portion 1032 may be substantially facing or opposite to surface portion 1011, which is fixed by adhesive layer 1002. Since surface portion 1011 is fixed, a large change in curvature may be found in surface portion 1011. Due to top layer 1004 being less compliant, a smaller change in curvature may be found near surface portion 1032. In some examples, smaller changes in curvature may result in a smoother surface at top layer 1004. For example, surface portion 1032 may create a substantially smooth surface between surface portions 1031 and 1033. In some examples, a shape of top layer 1004 may form a shape of an outer surface of an optical lens, which may be used to focus light rays at a point or may be used as corrective optics.

FIGS. 11A and 11B depict top views of examples of an adaptive optical material using one or more fluids, according to an embodiment. As shown, FIG. 11A depicts adaptive optical material 1100 including underlying substrate 1101, adhesive layer 1102, connection to pump (e.g., outlet) 1106, channel 1107, and bladder or bubble 1108. As shown, for example, adaptive optical material 1100 may have nine bladders 1108, including a larger central bladder, and eight side bladders having a substantially same size and located at a substantially same radius from the center. Other configurations may also be used. In some examples, elements 1102-1108 are transparent and may have a substantially same or similar index of refraction. Note, however, elements 1102-1108 are not limited to being transparent. In some examples, underlying substrate 1101 may be a substantially rigid material such as glass and may serve as a base or surface on which other layers may form. In some examples, underlying substrate 1101 may be octagonal (as shown), or may be another shape.

In some examples, adhesive layer 1102 may be formed over underlying substrate 1101 and may adhere portions of an intermediate layer to underlying substrate 1101. Portions of an intermediate layer that are not fixed to underlying substrate 1101 may form bladders 1108. Edges of adhesive layer 1102 may form perimeters of bladders 1108. Perimeters of bladders 1108 may be circular in shape (as shown) or another shape. Each bladder 1108 may be a same or different shape and size. In some examples, a configuration of bladders 1108 may be symmetrical around a center of underlying substrate 1101 or an intermediate layer (as shown). In other examples, other configurations may be used.

Bladders 1108 may be configured to receive a fluid through channels 1107. In some examples, channels 1107 may run from bladders 1108, through underlying substrate 1101, to outlets 1106. In other examples, channels 1107 may run differently. For example, outlets 1106 may be disposed at a bottom of underlying substrate 1101. In some examples, outlets 1106 may be connected to a pump or other device for inserting or draining a fluid. Each outlet 1106 may transfer to bladders 1108 a same or different fluids, which may have a same or different indices of refraction. A fluid may be, for example, a liquid or a gas, or another element or compound. For example, a fluid suitable for use in adaptive optical material 1100 may be water, which may have an index of refraction of 1.33 at 20 degrees Celsius. As another example, a fluid may be an oil, or any other material that may be pumped into bladder 1108.

As described above, bladders 1108 may be configured to receive a fluid through channels 1107. As a volume of a fluid received by bladder 1108 increases, bladder 1108 may expand. In some examples, as bladder 1108 expands, a volume or surface area of bladder 1108 may increase. As bladder 1108 expands, a distance between an intermediate layer and an underlying substrate forming bladder 1108 may increase. As bladder expands 1108, bladder 1108 and a fluid inside bladder 1108 may assert a pressure against a portion of an intermediate layer over bladder 1108. In other examples, reversibly, as fluid is drained, bladder 1108 may recoil, and a pressure against a portion of an intermediate layer over bladder 1108 may be reduced or removed. In some examples, a pressure may be applied against a portion of an intermediate layer that is substantially over bladder 1108 and is not be fixed to underlying substrate 1101 by adhesive layer 1102. In other examples, another portion of an intermediate layer that is fixed to underlying substrate 1101 by adhesive layer 1102 may be substantially restricted from expanding, contracting, or otherwise changing shape. Hence, some portions of an intermediate layer may be expanded, while other portions are not expanded, which may cause a surface of the intermediate layer to be substantially uneven, bumpy, or irregular.

As described above, a top layer may be formed above an intermediate layer. In some examples, a level of compliance of a top layer may be less than a level of compliance of an intermediate layer. A top layer may deform less than an intermediate layer when a same pressure is applied to both layers. Thus, a top layer may be configured to smoothen uneven edges or bumps of an intermediate layer. In some examples, a surface of a top layer (e.g., the surface of a top layer that is farther away from an intermediate layer) may be substantially smooth. In other examples, a change in slope of a surface of a top layer may be less than a change in slope of a surface of an intermediate layer. Curvatures or shapes of a top layer and an intermediate layer, a volume or shape of bladders 1108, and other factors may adaptively vary a focal length, a power, a diopter, an axis, or other parameter of an optical lens formed by adaptive optical material 1100.

FIG. 11B depicts adaptive optical material 1110 having bladders 1118a and 1118b. A variety of configurations of bladders may be used. As shown, for example, adaptive optical material 1110 includes a larger central bladder 1118a, and a number of side bladders (e.g., 1118b) having a variety of sizes. A size of side bladders may decrease as they reach a perimeter of adaptive optical material 1110. For example, central bladder 1118a may have a larger perimeter or radius than side bladder 1118b. Thus, a central bladder may receive a larger volume of fluid than a side bladder, resulting in a central bladder having a larger surface displacement than a side bladder. For example, a distance between a portion of an intermediate layer and a portion of an underlying substrate forming a larger central bladder 1118a may be able to expand more than a distance between another portion of an intermediate layer and another portion of an underlying substrate forming a smaller side bladder 1118b. This configuration may result in a convex lens, which may be used to correct myopia or other aberrations of an eye. In some examples, a larger number of bladders, a larger variety in sizes of bladders, and other configurations may increase a resolution with which a focal length or other parameter of adaptive optical material 1110 may be adaptively adjusted. For example, a parameter of adaptive optical material 1110 may be fine-tuned or adjusted by a larger number of bladders than adaptive optical material 1100 shown in FIG. 11A. Also as shown, for example, a location and shape of side bladders may be arranged symmetrically around a center of adaptive optical material 1110. In some examples, a symmetrical arrangement of bladders may result in an axis of adaptive optical material 1110 that intersects with a center of adaptive optical material 1110.

In some examples, adaptive optical material 1110 may include a smaller central bladder, and numerous side bladders that increase in size as they become farther from a center. Thus, a central bladder may receive a smaller volume of fluid than a side bladder, resulting in a central bladder having a smaller surface displacement than a side bladder. This configuration may result in a concave lens, which may be used to correct hyperopia or other aberrations of an eye. Still, other shapes, sizes, and configurations of bladders may be used.

In some examples, two or more adaptive optical materials 1110 may form a compound lens. A compound lens may include two or more adaptive optical materials 1110, and each adaptive optical material 1110 may have a different focal length, power, or other parameter, based on one or more volumes of fluid in one or more bladders of each adaptive optical material 1110. A compound lens may be used to improve optical corrections of aberrations of an eye (e.g., chromatic aberration, etc.). Still, other configurations of adaptive optical material may be used.

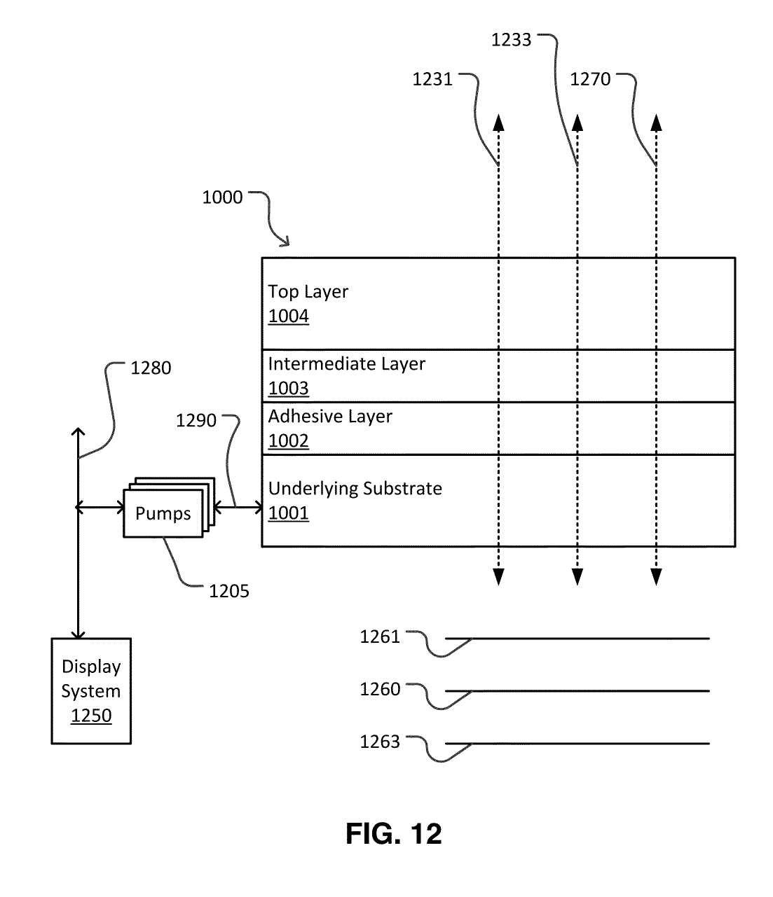

FIG. 12 depicts an example of an adaptive optical material using one or more fluids coupled to a control, according to an embodiment. As shown, FIG. 12 depicts adaptive optical material 1000 having an underlying substrate 1001, adhesive layer 1002, intermediate layer 1003, and top layer 1004. Adaptive optical material 1000, underlying substrate 1001, adhesive layer 1002, intermediate layer 1003, and top layer 1004 may be substantially similar structurally and functionally to similarly-named and numbered elements shown in FIG. 10. FIG. 12 also depicts one or more pumps 1205, display system 1250, light rays 1231, 1233, 1270, focal plane of a retinal surface of an eye 1260, myopia plane 1261, and hyperopia plane 1263.

In some examples, display system 1250 may be configured to transmit a control signal 1280 to one or more pumps 1205. Based on control signal 1280, pumps 1205 may inject or drain a fluid through channel 1290 to or from adaptive optical material 1000. A fluid from pumps 1205 may be received by one or more bladders of adaptive optical material 1000. Each pump 1205 may be individually controlled by display system 1250, and may be individually coupled to a bladder. As described above, a focal length, power, or other parameter of adaptive optical material 1000 may be adaptively changed as a function of a volume of fluid received.

In some examples, display system 1250 may be configured to change a parameter of adaptive optical material 1000 based on a detection of an aberration of an eye. Focal plane of a retinal surface 1060 may represent a plane in which light rays (e.g., 1231, 1233, and 1270) converge without the use of corrective optics for an ideal eye (e.g., an eye with no aberration). Myopia plane 1261 may represent a plane in which light rays (e.g., 1231, 1233, and 1270) converge without the use of corrective optics for an eye with myopia, and hyperopia plane 1263 may represent a plane in which light rays (e.g., 1231, 1233, and 1270) converge without the use of corrective optics for an eye with hyperopia. Light rays may also converge on other planes (not shown) due to other aberrations (e.g., astigmatism).

In some examples, display system 1250 may include an image projector (not shown) to form light ray 1031 to project an image to a portion of an eye. In other examples, display system 1250 may include an image capture device (not shown) to receive light ray 1033 from a reflected image from a portion of an eye. Delivery optics including a mirror, beam splitter, prism, or the like may be used to optically couple display system 1250 (e.g., image projector, image capture device, etc.) and a portion of an eye. In some examples, light ray 1070 may also be generated from an ambient light, which may project an ambient image to a portion of an eye. Light rays 1231, 1233, and 1270 may pass through adaptive optical material 1000 to reach a portion of an eye. In some examples, display system 1250 may compare an image projected by an image projector (e.g., a projected image) and an image received by an image capture device (e.g., a reflected image), and determine a difference between the images. Based on a difference between the images, display system 1250 may send a control signal 1280 to adjust pumps 1205 to adaptively change one or more volumes of fluid received by one or more bladders of adaptive optical material 1000. Display system 1250 may adjust pumps 1205 until a difference between a projected image and a reflected image is reduced, minimized, or eliminated. In some examples, one or more adaptive optical materials 1000 may be adjusted by display system 1250, including forming a spherical lens, forming a cylindrical lens, or adjusting a spherical power, cylindrical power, cylindrical axis orientation, or other parameters. In other examples, display system 1250 may also diagnose or determine an aberration of an eye, such as a degree of myopia or hyperopia, or the like, based on a difference between a projected image and a reflected image, based on adjustments made to adaptive optical material 1000 to minimize the difference, or based on other factors.

In still other examples, display system 1250 may be manually controlled. Display system 1250 may receive a manual input, which may describe an aberration of an eye, describe a parameter of a lens to be achieved, describe one or more volumes of fluid to be received by one or more bladders, or the like. Display system 1250 may adjust pumps 1205 based on the manual input. For example, display system 1250 may be a conventional pump, which may be manually controlled to inject or drain a volume of fluid to or from a bladder. Still, other implementations of display system 1250 may be used.

FIGS. 13A and 13B depict cross-sectional views of examples of an adaptive optical material having a shape modified by one or more fluids, according to an embodiment. As shown, FIG. 13A depicts adaptive optical material 1300 including underlying substrate 1301, adhesive layer 1302, intermediate layer 1303, top layer 1304, and bladders 1308a and 1308b. As described above, in some examples, intermediate layer 1304 may be a flexible material that may expand or contract based on an amount of pressure applied against it. Portions of intermediate layer 1304 may be fixed to underlying substrate 1301 using adhesive layer 1302. Portions of intermediate layer 1304 that are not fixed to underlying substrate 1301 by adhesive layer 1302 may form bladders 1308a and 1308b. Bladders 1308a and 1308b may be of different sizes or shapes. In some examples, bladders 1308a and 1308b may be configured to receive a fluid. As a volume of fluid received increases, a thickness of bladders 1308a and 1308b may increase. Bladders 1308a and 1308b may assert pressure against portions of intermediate layer 1304 covering bladders 1308a and 1308b, causing these portions to expand. As shown, for example, intermediate layer 1304 may become uneven, bumpy, or irregular. As portions of intermediate layer 1304 push upward, they assert pressure against top layer 1304. In some examples, top layer 1304 may also be a flexible material that expands or contracts based on a pressure applied against it. In some examples, a level of compliance of top layer 1304 may be less than a level of compliance of intermediate layer 1303. Thus, top layer 1304 may deform less than intermediate layer 1303 when a substantially similar pressure is applied to both layers. As shown, for example, top layer 1304 may smoothen bumps in intermediate layer 1303. A top surface of top layer 1304 may be substantially smooth. Elements 1301-1304, 1308a and 1308b, form adaptive optical material 1300, which may be configured to focus collimated light rays at a point, and to adaptively change a distance between this point and adaptive optical material 1300 based on one or more volumes of fluid received by bladders 1308a and 1308b, which is further discussed herein (e.g., FIG. 13B). Still, other layers, portions of layers, or materials may be included in adaptive optical material 1300.

As shown, FIG. 13B includes adaptive optical material 1300, collimated light rays 1311a, 1311b, and 1311b, point 1312, and distance 1313. Adaptive optical material 1300 may be configured to serve as an optical lens. In some examples, adaptive optical material 1300 may focus collimated light rays 1311a, 1311b, and 1311c at point 1312 (e.g., focal point). Point 1312 may have a distance 1313 from a bottom of an underlying substrate of adaptive optical material 1300. Based on one or more volumes of fluid received by one or more bladders of adaptive optical material 1300, a focusing of collimated light rays 1311a, 1311b, and 1311c at point 1312, a distance 1313, and other parameters of adaptive optical material 1300 may be adjusted or controlled. For example, distance 1313 may be controlled such that point 1312 falls on a retinal plane of an eye when adaptive optical material 1300 is being worn, and adaptive optical material 1300 may serve as a corrective lens.

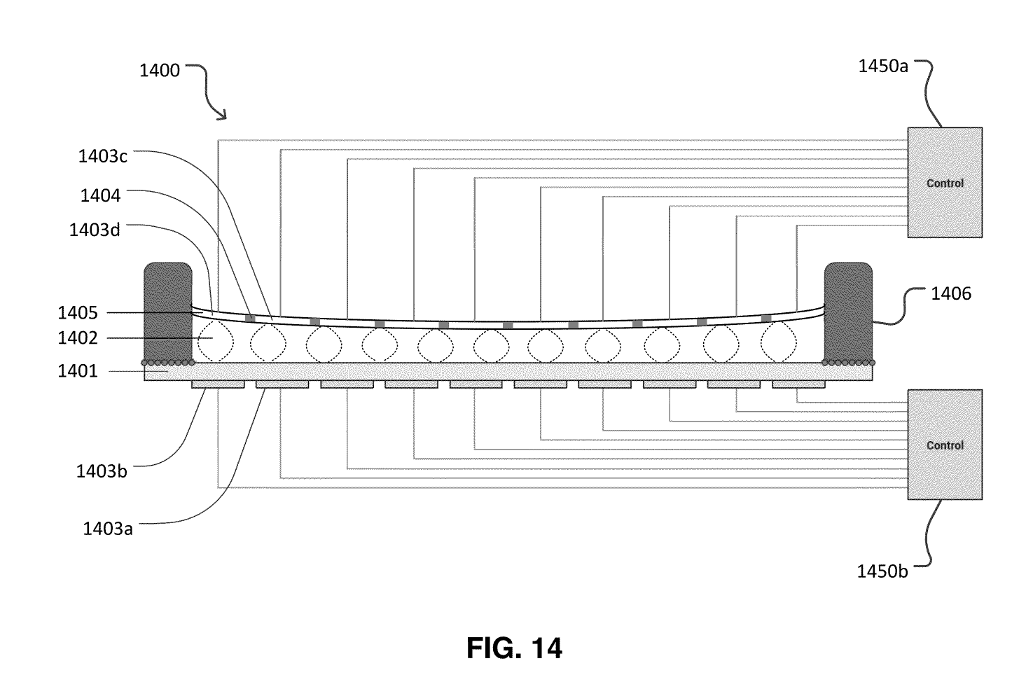

FIG. 14 depicts a cross-sectional view of an example of adaptive optical material using capacitance, according to an embodiment. As shown, for example, FIG. 14 depicts adaptive optical material 1400 including an underlying substrate 1401 having electrodes 1403a and 1403b, insulating layer 1402, flexible top layer 1405 having electrodes 1403c and 1403d and flexible insulator 1404, frame 1406, and control 1450a and 1450b. Adaptive optical material 1400 may be configured to focus light rays at a point and to change a distance between this point and the adaptive optical material using one or more voltages applied to one or more electrodes (e.g., 1403a, 1403b, 1403c, 1403d, etc.). Electrodes (e.g., 1403a, 1403b, 1403c, 1403d, etc.) may be a transparent conducting material (e.g., indium tin oxide, etc.). In some examples, electrodes (e.g., 1403a and 1403b) may be formed on a substrate, layer, or surface (e.g., underlying substrate 1401). In other examples, electrodes (e.g., 1403c and 1403d) may be formed as part of a conducting layer (e.g., top flexible layer 1405). An insulator (e.g., 1404) may be inserted in a layer of conducting material (e.g., top flexible layer 1405) to form numerous electrodes (e.g., 1403c and 1403d) in the conducting material. For example, numerous insulators may be disposed in top flexible layer 1405 to create numerous electrodes (e.g., 1403c and 1403d), such as an array or grid of electrodes. Further, electrodes may be flexible or rigid. In some examples, underlying substrate 1401 and electrodes 1403a and 1403b may be substantially rigid. In some examples, flexible top layer 1405 and electrodes 1403c and 1403d may be substantially flexible. Insulator 1404 may also be flexible. Flexible top layer 1405 and electrodes 1403c and 1403d may expand or contract based on a pressure applied against them. In some examples, numerous electrodes may be coupled to underlying substrate 1401 and numerous electrodes may be coupled to flexible top layer 1405 (as shown). In some examples, an electrode coupled to underlying substrate 1401 and another electrode coupled to flexible top layer 1405 may be substantially opposite from each other and may form a pair (e.g., electrodes 1403a and 1403c may form a pair, electrodes 1403b and 1403d may form a pair, etc.). Voltage potentials between pairs of electrodes having varying magnitudes and signs across a cross-section of adaptive optical material 1400 may be generated. In other examples, one electrode may be coupled to underlying substrate 1401 and numerous electrodes may be coupled to flexible top layer 1405. Voltage potentials between a single electrode coupled to underlying substrate 1401 and numerous electrodes coupled to top flexible layer 1405 having various magnitudes and signs across a cross-section of adaptive optical material 1400 may be generated by applying various voltages to one or more electrodes coupled to top flexible layer 1405. In some examples, variable voltages may be applied to electrodes coupled to underlying substrate 1401 and coupled to flexible top layer 1405. In other examples, a fixed voltage (e.g., ground, etc.) may be applied to electrodes coupled to underlying substrate 1401, or may be applied to electrodes coupled to flexible top layer 1405. Still, other configurations of electrodes may be used.

As described above, a voltage or electric signal may be applied to one or more electrodes (e.g., 1403a, 1403b, 1403c, 1403d, etc.), which may result in a voltage potential across an electrode (e.g., 1403a, 1403b, etc.) coupled to underlying substrate 1401 and another electrode (e.g., 1403c, 1403d, etc.) coupled to flexible top layer 1405. A voltage potential across electrodes may generate a force or pressure against a portion of underlying substrate 1401 or flexible top layer 1405. For example, a voltage potential across a pair of electrodes (e.g., 1403a and 1403c) may cause a portion of flexible top layer 1405 on which an electrode (e.g., 1403c) is formed to be pulled towards underlying substrate 1401, to expand or contract, or otherwise to deform or change in shape. One or more voltages may be individually controlled and applied to one or more electrodes by control 1450a and 1450b, and a shape of different portions of flexible top layer 1405 may be individually adjusted. For example, control 1450a and 1450b may apply a greater voltage to electrodes near a center of adaptive optical material 1400 and a lesser voltage to electrodes near a side of adaptive optical material 1400. As shown, for example, due to different voltages being applied to different electrodes, central portions of flexible top layer 1405 may be pulled more towards underlying substrate 1401, while side portions of flexible top layer 1405 may be pulled less towards underlying substrate 1401. As a result, flexible top layer 1405 may form a concave shape. Still, other shapes may be formed based on one or more voltages being applied to one or more electrodes.

Insulating layer 1402 may be a transparent, substantially non-conductive, and flexible material, such as a polymer (and may or may not be an electroactive polymer). Insulating layer 1402 may expand or contract or otherwise change shape based on a pressure applied against it. Insulating layer 1402 may disposed between underlying substrate 1401 and flexible top layer 1405 and may expand or contract based on the shapes of underlying substrate 1401 and flexible top layer 1405. In some examples, insulating layer 1402 may be adhered to underlying substrate 1401 and flexible top layer 1405. As shown, for example, as flexible top layer 1405 changes shape, insulating layer 1402 also changes shape. As shown, for example, substantially near a center of adaptive optical material 1400, flexible top layer 1405 may be pulled towards underlying substrate 1401, resulting in a lesser thickness between flexible top layer 1405 and underlying substrate 1401. Insulating layer 1402 may also contract, and become less thick near this region. Different portions or regions of insulating layer 1402 may be controlled individually, based on individual voltages applied to one or more electrodes along underlying substrate 1401 or flexible top layer 1405. In some examples, adaptive optical material 1400 may form a concave lens (as shown), a convex lens, or another shape or lens type. Adaptive optical material 1400 may focus incoming light to a point (e.g., a focal point) and may adjust a distance between this point and adaptive optical material 1400 (e.g., e.g., focal length) based on a curvature of top flexible layer 1405, which may be controlled by voltages applied to one or more electrodes. Adaptive optical material 1400 may be used as a corrective lens, to substantially correct aberrations of the eye. Still, other layers, portions of layers, or materials may be used in adaptive optical material 1400, and an order of layers may be different. For example, one or both of control 1450a and 1450b may not be included, frame 1406 may be different or may not be included, a shape or curvature of underlying substrate 1401 may be different, and the like.

In some examples, control 1450a and 1450b may be configured to individually control one or more voltages applied to one or more electrodes (e.g., 1403a, 1403b, 1403c, 1403d, etc.). As shown, for example, control 1450a and 1450b may be electrically coupled to each individual electrode. In other examples, other control configurations may be used. For example, control 1450a may be coupled to electrodes formed on top flexible layer 1405, while a fixed voltage (e.g., ground, etc.) may be coupled to electrodes formed on underlying substrate 1401. In some examples, control 1450a and 1450b may be coupled to an image projector and an image capture device. As described above, an image projector (not shown) may project an image through adaptive optical material 1400 to a portion of an eye, and an image capture device (not shown) may receive a reflected image through adaptive optical material 1400 from a portion of an eye. Delivery optics including a mirror, beam splitter, prism, or the like may be used to optically couple an image projector and an image capture device to a portion of an eye. In some examples, control 1450a and 1450b may compare an image projected by an image projector (e.g., a projected image) and an image received by an image capture device (e.g., a reflected image), and determine a difference between the images. Based on a difference between the images, control 1450a and 1450b may adjust one or more voltages applied to one or more electrodes (e.g., 1403a, 1403b, 1403c, 1403d, etc.) of adaptive optical material 1400. In some examples, control 1450a and 1450b may adjust voltages applied until a difference between a projected image and a reflected image is reduced, minimized, or eliminated. In some examples, control 1450a and 1450b may be coupled to one or more adaptive optical materials 1400, which may form a compound lens, and may adjust a spherical power, cylindrical power, a cylindrical axis orientation, or other parameters of the compound lens. In still other examples, control 1450a and 1450b may be manually controlled, based on user input that may specify one or more voltages to be applied, a parameter to be achieved by adaptive optical material 1400, and the like.

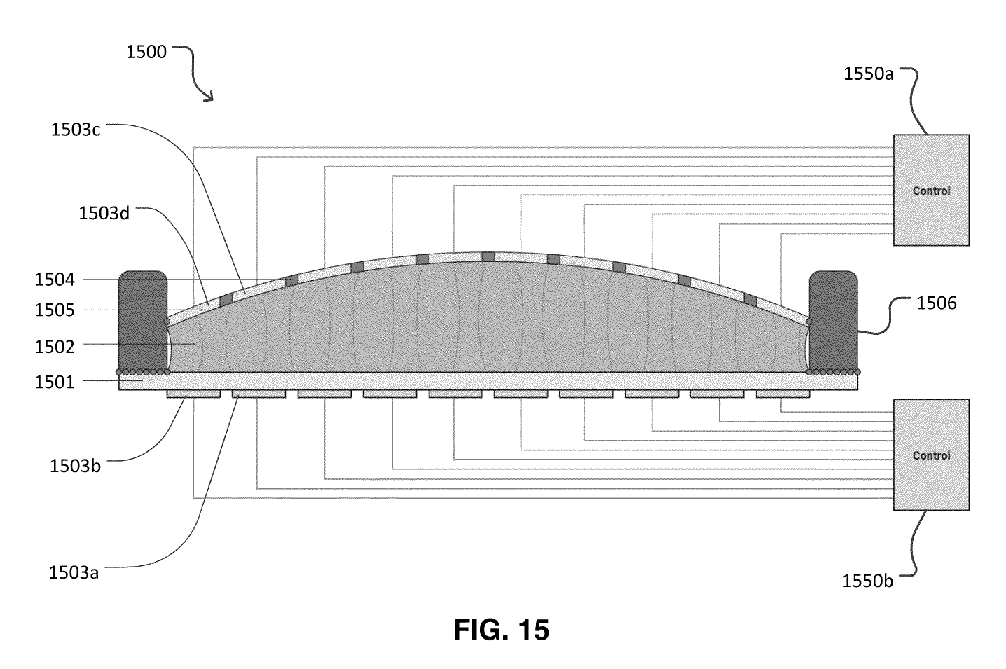

FIG. 15 depicts a cross-sectional view of an example of adaptive optical material using electroactive material, according to an embodiment. As shown, for example, FIG. 15 depicts adaptive optical material 1500 including an underlying substrate 1501 having electrodes 1503a and 1503b, electroactive layer 1502, flexible top layer 1505 having electrodes 1503c and 1503d and flexible insulator 1504, frame 1506, and control 1550a and 1550b. Adaptive optical material 1500 may be configured to focus light rays at a point and to change a distance between this point and the adaptive optical material using one or more voltages applied to one or more electrodes (e.g., 1503a, 1503b, 1503c, 1503d, etc.). Electrodes (e.g., 1503a, 1503b, 1503c, 1503d, etc.) may be a transparent conducting material (e.g., indium tin oxide, etc.). In some examples, electrodes (e.g., 1503a and 1503b) may be formed on a substrate, layer, or surface (e.g., underlying substrate 1501). In other examples, electrodes (e.g., 1503c and 1503d) may be formed as part of a conducting layer (e.g., top flexible layer 1505). An insulator (e.g., 1504) may be inserted in a layer of conducting material (e.g., top flexible layer 1505) to form numerous electrodes (e.g., 1503c and 1503d) in the conducting material. For example, numerous insulators may be disposed in top flexible layer 1505 to create numerous electrodes (e.g., 1503c and 1503d), such as an array or grid of electrodes. Further, electrodes may be flexible or rigid. In some examples, underlying substrate 1501 and electrodes 1503a and 1503b may be substantially rigid. In some examples, flexible top layer 1505 and electrodes 1503c and 1503d may be substantially flexible. Insulator 1504 may also be flexible. Flexible top layer 1505 and electrodes 1503c and 1503d may expand or contract based on a pressure applied against them. In some examples, numerous electrodes may be coupled to underlying substrate 1501 and numerous electrodes may be coupled to flexible top layer 1505 (as shown). In some examples, an electrode coupled to underlying substrate 1401 and another electrode coupled to flexible top layer 1405 may be substantially opposite each other and form a pair (e.g., electrodes 1503a and 1503c may form a pair, electrodes 1503b and 1503d may form a pair, etc.). Varying voltage potentials may be generated across various pairs of electrodes. In other examples, one electrode may be coupled to underlying substrate 1501 and numerous electrodes may be coupled to flexible top layer 1505. Voltage potentials between a single electrode coupled to underlying substrate 1501 and numerous electrodes coupled to top flexible layer 1505 having various magnitudes and signs across a cross-section of adaptive optical material 1500 may be generated by applying various voltages to one or more electrodes coupled to top flexible layer 1505. In some examples, variable voltages may be applied to electrodes coupled to underlying substrate 1501 and coupled to flexible top layer 1505. In other examples, a fixed voltage (e.g., ground, etc.) may be applied to electrodes coupled to underlying substrate 1501, or may be applied to electrodes coupled to flexible top layer 1505. Still, other configurations of electrodes may be used.

As described above, a voltage or electric signal may be applied to one or more electrodes (e.g., 1503a, 1503b, 1503c, 1503d, etc.), which may result in a voltage potential across an electrode (e.g., 1503a, 1503b, etc.) coupled to underlying substrate 1501 and another electrode (e.g., 1503c, 1503d, etc.) coupled to flexible top layer 1505. One or more voltages may be individually controlled and applied to one or more electrodes by control 1550a and 1550b. For example, control 1550a and 1550b may apply a greater voltage to electrodes near a center of adaptive optical material 1500 and a lesser voltage to electrodes near a side of adaptive optical material 1500. A voltage potential across electrodes may create an electric field across the electrodes. An electric field may cause electroactive layer 1502 to expand or contract.

In some examples, electroactive layer 1502 may be a transparent flexible material (e.g., an electroactive polymer) disposed between underlying substrate 1501 and top flexible layer 1505. In some examples, electroactive layer 1502 may be adhered to underlying substrate 1501 and flexible top layer 1505. Electroactive layer 1502 may be configured to change in size or shape when a voltage or electric field is applied across it. For example, a voltage potential across a pair of electrodes (e.g., 1503a and 1503c) may cause a portion of electroactive layer 1502 disposed substantially between the pair of electrodes to expand or contract, or otherwise deform or change in shape. Various portions of electroactive layer 1502 may expand or contract based on various voltages applied to various electrodes. As portions of electroactive layer 1502 expand or contract, various pressures may be asserted against flexible top layer 1505, which may cause portions of flexible top layer 1505 to expand or contract. As shown, for example, due to different voltages being applied to different electrodes, central portions of flexible top layer 1505 may expand more, while side portions of flexible top layer 1505 may expand less. As a result, flexible top layer 1505 may form a convex shape. Still, other shapes may be formed based on one or more voltages being applied to one or more electrodes.

In some examples, adaptive optical material 1500 may form a concave lens, a convex lens (as shown), or another shape or lens type. Adaptive optical material 1500 may focus incoming light to a point (e.g., a focal point) and may adjust a distance between this point and adaptive optical material 1500 (e.g., e.g., focal length) based on a curvature of top flexible layer 1505, which may be controlled by voltages applied to one or more electrodes. Adaptive optical material 1500 may be used as a corrective lens, to substantially correct aberrations of the eye. Still, other layers, portions of layers, or materials may be used in adaptive optical material 1500, and an order of layers may be different. For example, one or both of control 1550a and 1550b may not be included, frame 1506 may be different or may not be included, a shape or curvature of underlying substrate 1501 may be different, and the like.

In some examples, control 1550a and 1550b may be configured to individually control one or more voltages applied to one or more electrodes (e.g., 1503a, 1503b, 1503c, 1503d, etc.). As shown, for example, control 1550a and 1550b may be electrically coupled to each individual electrode. In other examples, other control configurations may be used. For example, control 1550a may be coupled to electrodes formed on top flexible layer 1505, while a fixed voltage (e.g., ground, etc.) may be coupled to electrodes formed on underlying substrate 1501. In some examples, control 1550a and 1550b may be coupled to an image projector and an image capture device. As described above, an image projector (not shown) may project an image through adaptive optical material 1500 to a portion of an eye, and an image capture device (not shown) may receive a reflected image through adaptive optical material 1500 from a portion of an eye. Delivery optics including a mirror, beam splitter, prism, or the like may be used to optically couple an image projector and an image capture device to a portion of an eye. In some examples, control 1550a and 1550b may compare an image projected by an image projector (e.g., a projected image) and an image received by an image capture device (e.g., a reflected image), and determine a difference between the images. Based on a difference between the images, control 1550a and 1550b may adjust one or more voltages applied to one or more electrodes (e.g., 1503a, 1503b, 1503c, 1503d, etc.) of adaptive optical material 1500. In some examples, control 1550a and 1550b may adjust voltages applied until a difference between a projected image and a reflected image is reduced, minimized, or eliminated. In some examples, control 1550a and 1550b may be coupled to one or more adaptive optical materials 1500, which may form a compound lens, and may adjust a spherical power, cylindrical power, a cylindrical axis orientation, or other parameters of the compound lens. In still other examples, control 1550a and 1550b may be manually controlled, based on user input that may specify one or more voltages to be applied, a parameter to be achieved by adaptive optical material 1500, and the like.

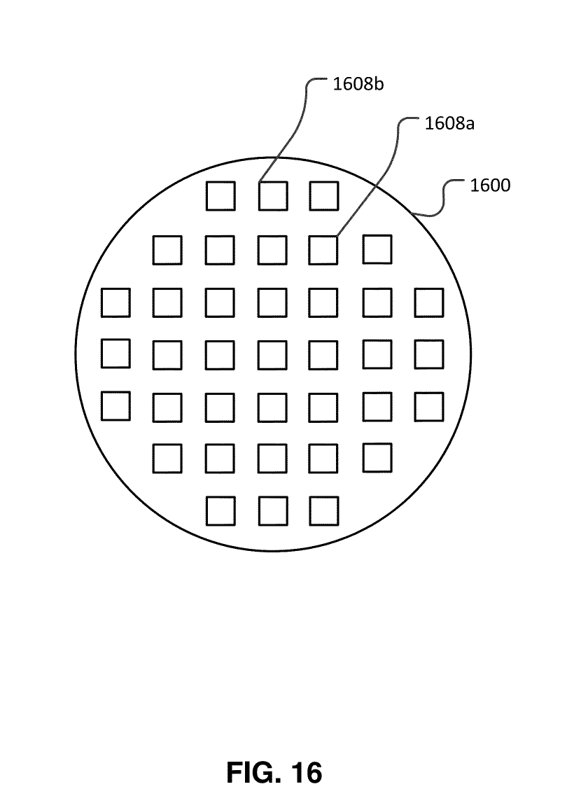

FIG. 16 depicts a top view of an example of an adaptive optical material using an electrode grid, according to an embodiment. Here, adaptive optical material 1600 may include numerous electrodes (e.g., 1608a and 1608b). Adaptive optical material 1600 may be used with adaptive optical material 1400 shown in FIG. 14, adaptive optical material 1500 shown in FIG. 15, or other adaptive optical materials using electrodes. For example, while FIGS. 14 and 15 illustrate an array of electrodes across a cross-section of an adaptive optical material, these arrays may extend across the adaptive optical material to form a grid, such as the grid shown in FIG. 16. A grid may include a number of rows and columns of electrodes. In other examples, electrodes may be placed in other configurations, such as in a circular, elliptical, or irregular fashion. Other configurations of electrodes may be used.

Electrodes (e.g., 1608a and 1608b) may be coupled to a top flexible layer of adaptive optical material 1600, and may be configured to receive an electric signal to generate a voltage potential between the top flexible layer and an underlying substrate. In some examples, individual electrodes 1608a and 1608b may be formed on a surface of a top flexible layer. In other examples, a top flexible layer may be a conducting material, and insulators may be added on or in the top flexible layer to create individual electrodes 1608a and 1608b. Electrodes 1608a and 1608b may take any shape or size, including square, rectangular, circular, irregular, and the like. Electrodes 1608a and 1608b may be a same or different shape or size. Electrodes 1608a and 1608b may be disposed symmetrically (as shown) or asymmetrically about a center of adaptive optical material 1600. Still, other shapes, sizes, and configurations of electrodes may be used.

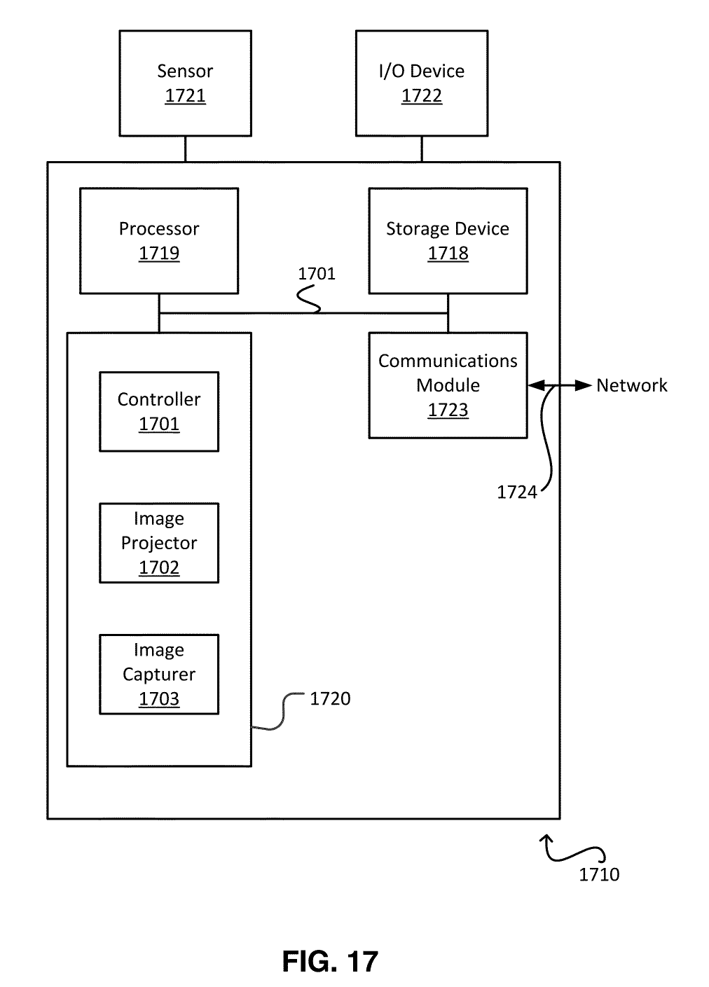

FIG. 17 depicts a computer system suitable for use with an adaptive optical material, according to an embodiment. In some examples, computing platform 1710 may be used to implement computer programs, applications, methods, processes, algorithms, or other software to perform the above-described techniques. Computing platform 1710 includes a bus 1701 or other communication mechanism for communicating information, which interconnects subsystems and devices, such as processor 1719, system memory 1720 (e.g., RAM, etc.), storage device 1718 (e.g., ROM, etc.), a communications module 1723 (e.g., an Ethernet or wireless controller, a Bluetooth controller, etc.) to facilitate communications via a port on communication link 1724 to communicate, for example, with a computing device, including mobile computing and/or communication devices with processors. Processor 1719 can be implemented with one or more central processing units (“CPUs”), such as those manufactured by Intel® Corporation, or one or more virtual processors, as well as any combination of CPUs and virtual processors. Computing platform 1710 exchanges data representing inputs and outputs via input-and-output devices 1722, including, but not limited to, keyboards, mice, audio inputs (e.g., speech-to-text devices), speakers, microphones, user interfaces, displays, monitors, cursors, touch-sensitive displays, LCD or LED displays, and other I/O-related devices. An interface is not limited to a touch-sensitive screen and can be any graphic user interface, any auditory interface, any haptic interface, any combination thereof, and the like. Computing platform 1710 may also receive sensor data from sensor 1721, including a heart rate sensor, a respiration sensor, an accelerometer, a motion sensor, a galvanic skin response (GSR) sensor, a bioimpedance sensor, a GPS receiver, and the like.

According to some examples, computing platform 1710 performs specific operations by processor 1719 executing one or more sequences of one or more instructions stored in system memory 1720, and computing platform 1710 can be implemented in a client-server arrangement, peer-to-peer arrangement, or as any mobile computing device, including smart phones and the like. Such instructions or data may be read into system memory 1720 from another computer readable medium, such as storage device 1718. In some examples, hard-wired circuitry may be used in place of or in combination with software instructions for implementation. Instructions may be embedded in software or firmware. The term “computer readable medium” refers to any tangible medium that participates in providing instructions to processor 1719 for execution. Such a medium may take many forms, including but not limited to, non-volatile media and volatile media. Non-volatile media includes, for example, optical or magnetic disks and the like. Volatile media includes dynamic memory, such as system memory 1720.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1001 for transmitting a computer data signal.

In some examples, execution of the sequences of instructions may be performed by computing platform 1710. According to some examples, computing platform 1710 can be coupled by communication link 1724 (e.g., a wired network, such as LAN, PSTN, or any wireless network) to any other processor to perform the sequence of instructions in coordination with (or asynchronous to) one another. Computing platform 1710 may transmit and receive messages, data, and instructions, including program code (e.g., application code) through communication link 1724 and communication interface 1723. Received program code may be executed by processor 1719 as it is received, and/or stored in memory 1720 or other non-volatile storage for later execution.

In the example shown, system memory 1720 can include various modules that include executable instructions to implement functionalities described herein. In the example shown, system memory 1720 includes a controller 1701, an image projector 1702, and an image capturer 1703.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed is:

1. An device, comprising:
a layer;
a substrate; and
an intermediate layer disposed between the substrate and the layer, the intermediate layer comprising:
a first bladder including a first surface portion associated with the intermediate layer and a second surface portion associated with the substrate, the first bladder configured to receive a first volume of fluid to form a first distance between the first surface portion and the second surface portion;
a second bladder including a third surface portion associated with the intermediate layer and a fourth surface portion associated with the substrate, the second bladder configured to receive a second volume of fluid to form a second distance between the third surface portion and the fourth surface portion, the second distance being different than the first distance,
wherein a portion of a surface of the layer may be configured to have a degree of curvature relative to a line perpendicular to the substrate based on a difference between the first distance and the second distance, the degree of curvature configured to focus a subset of collimated light rays substantially at a point.

2. The device of claim 1, further comprising:
an adhesive layer adhering a portion of the intermediate layer to another portion of the substrate to form the first bladder and the second bladder.

3. The device of claim 1, further comprising:
an outlet configured to receive the first volume of fluid; and
a channel extending from the outlet to the first bladder, at least a portion of the channel running through the substrate.

4. The device of claim 1, further comprising:
a first pump configured to control the first volume of fluid; and
a second pump configured to control the second volume of fluid.

5. The device of claim 1, wherein the degree of curvature may be further configured to modify a distance between the point and the substrate based on the first volume of fluid and the second volume of fluid.

6. The device of claim 1, wherein a level of compliance associated with the layer is less than another level of compliance associated with the intermediate layer.

7. The device of claim 1, wherein the first bladder is located substantially at a center of the intermediate layer and the second bladder is located at a radius from the center of the intermediate layer, a first perimeter of the first bladder being greater than a second perimeter of the second bladder.

8. The device of claim 1, further comprising:
a third bladder including a fifth surface portion associated with the layer and a sixth surface portion associated with the substrate, the third bladder configured to receive a third volume of fluid to form a third distance between the fifth surface portion and the sixth surface portion,
wherein the second bladder and the third bladder are located substantially at a same radius from a center of the intermediate layer and have perimeters that are substantially equal.

9. The device of claim 1, further comprising:
an image projector configured to project an image through the substrate, the intermediate layer, and the layer to a portion of an eye;
an image capture device configured to receive another image through the substrate, the intermediate layer, and the layer reflected from the portion of the eye; and
a control system configured to perform a comparison of the image and the another image, and to control the first volume of liquid and the second volume of liquid based on the comparison.

10. The device of claim 1, further comprising:
a chassis physically coupled to at least one of the substrate, the intermediate layer, and the layer, and configured to be worn.

11. The device of claim 1, wherein the indices of refraction of the intermediate layer and the fluid are substantially equal.

12. A device, comprising:
a substrate having one or more electrodes;
a flexible layer having one or more other electrodes, the flexible layer configured to change in shape as a function of one or more voltages applied to the one or more other electrodes; and
an insulating layer disposed between the substrate and the flexible layer,
wherein the device is configured to focus a plurality of collimated light rays substantially at a point and to modify a distance between the point and a surface of the flexible layer as a function of the one or more voltages.

13. The device of claim 12, wherein the one or more other electrodes form a grid.

14. The device of claim 12, further comprising:
a control system configured to individually control the one or more voltages applied to the one or more other electrodes.

15. The device of claim 12, further comprising:
an image projector configured to project an image through the substrate, the insulating layer, and the flexible layer to a portion of an eye;
an image capture device configured to receive another image through the substrate, the insulating layer, and the flexible layer reflected from the portion of the eye; and
a control system configured to perform a comparison of the image and the another image, and to individually control the one or more voltages applied to the one or more other electrodes based on the comparison.

16. The device of claim 12, further comprising:
a chassis physically coupled to at least one of the substrate, the flexible layer, and the insulating layer, and configured to be worn.

17. A device, comprising:
a substrate having one or more electrodes;
a flexible layer having one or more other electrodes; and
an electroactive layer disposed between the substrate and the flexible layer and having a plurality of regions, each region being disposed between one electrode of the one or more electrodes and another electrode of the one or more other electrodes and being configured to change in volume as a function of a voltage applied to the another electrode,
wherein the device is configured to focus a plurality of collimated light rays substantially at a point and to modify a distance between the point and a surface of the flexible layer as a function of the voltage.

18. The device of claim 17, wherein the one or more other electrodes form a grid.

19. The device of claim 17, further comprising:
a control system configured to individually control one or more voltages applied to the one or more other electrodes.

20. The device of claim 17, further comprising:
an image projector configured to project an image through the substrate, the electroactive layer, and the flexible layer to a portion of an eye;
an image capture device configured to receive another image through the substrate, the electroactive layer, and the flexible layer reflected from the portion of the eye; and
a control system configured to perform a comparison of the image and the another image, and to individually control one or more voltages applied to the one or more other electrodes.
